(19) United States
(12) Patent Application Publication
(10) Pub. No.: US 2024/0000838 A1
(43) Pub. Date: Jan. 4, 2024

(54) UNIVERSAL CHIMERIC ANTIGEN RECEPTOR-EXPRESSING IMMUNE CELLS FOR ALLOGENEIC CELL THERAPY

(71) Applicant: SIMCERE INNOVATION, INC., Cambridge, MA (US)

(72) Inventors: Daofeng LIU, Cambridge, MA (US); Pei-Yun TSAI, Cambridge, MA (US); Zhongdong SHI, Cambridge, MA (US); Xiangbin XU, Cambridge, MA (US)

(21) Appl. No.: 18/255,095

(22) PCT Filed: Nov. 24, 2021
(86) PCT No.: PCT/US2021/072602

§ 371 Date: May 30, 2023

Related U.S. Application Data
(60) Provisional application No. 63/119,227, filed on Nov. 30, 2020.

(51) Int. Cl. A61K 35/17; C12N 5/0783; A61K 39/00; C12N 15/86; A61P 35/02

(52) U.S. Cl. CPC A61K 35/17; C12N 5/0646; A61K 39/4613; A61K 39/4631; C12N 15/86; A61P 35/02; A61K 2239/21; A61K 2239/17; A61K 2239/48; A61K 2239/25

(57) ABSTRACT

Compositions and methods for treating diseases associated with expression of cluster differentiation 33 (CD33) and/or cluster differentiation 5 (CD5) involve two chimeric antigen receptors (CARs) specific to CD33 and CD5 and T cells with CD33 and CD5 dual-CAR. Methods of administering a genetically modified T cell expressing the dual-CAR can be used for autologous and allogeneic treatment of T cell malignancies.

### RELATED APPLICATIONS

This application claims the benefit of U.S. Provisional Application No. 63/119,227, filed on Nov. 30, 2020. The entire teachings of the above application is incorporated herein by reference.

### INCORPORATION BY REFERENCE OF MATERIAL IN ASCII TEXT FILE

This application incorporates by reference the Sequence Listing contained in the following ASCII text file being submitted concurrently herewith:
a) File name: 58011002002_SEQUENCELISTING.txt; created Oct. 5, 2021, 139,353 Bytes in size.

### BACKGROUND

Graft-versus-host disease (GVHD) can be an impediment to effective allogeneic cell therapy. Most T cells (>90%) in the human body express a T cell receptor (TCR) heterodimer comprising an α chain and β chain and are referred to as αβT cells. If using αβT cells for allogeneic cell therapy, the endogenous TCR on these allogeneic αβT cells may recognize the alloantigens of the recipients through a major histocompatibility complex (MHC)-dependent pathway, leading to GVHD; furthermore, the expression of HLA on the surface of allogeneic T cells may cause rapid rejection by the host immune system (host-versus graft disease, or HVGD). Other types of immune cells, including γδT, NKT and NK cells, may be used for allogeneic therapy, but there are clear disadvantages for each of them. Therefore, a simple and efficient method of generating universal immune cells containing chimeric antigen receptors (CARs) for allogeneic therapy is to genetically modify αβT cells. The early generation of universal CAR-T cells (UCAR-T) was made by knocking out the TCR α chain and/or β chain to prevent GVHD, and either B2M or HLA-I molecules to avoid rejection by HVG. However, removal or reduction of HLA-I expression can elicit strong killing by NK cells. To reduce NK killing of these cells, HLA-E, HLA-F, or HLA-G has been ectopically expressed, but the resulting inhibition of NK killing is very limited.

Cellectis and Allogene have designed an alternative strategy to produce UCAR-T using αβT cells in combination with a CD52 monoclonal antibody, alemtuzumab. Using a genome editing tool—transcription activator-like effector nuclease (TALEN) technology, the TCR and CD52 in αβT cells were both knocked out to prevent GVHD and to resist toxicity of alemtuzumab on UCAR-T cells, respectively. Preliminary results from Phase 1 studies of UCAR-T against CD19 positive relapsed/refractory acute lymphoblastic leukemia (ALL) show 82% complete remission rate across the adult and pediatric patient population who received a lymphodepletion regimen consisting of fludarabine, cyclophosphamide and alemtuzumab. However, more than 70% these patients relapsed or died within a year even after allogeneic stem cell transplantation.

### SUMMARY

One problem with prior processes is that they can require 3 to 4 weeks, which might be too long for some patients whose diseases progress very rapidly. In some cases, the patients' T cells may not be in good quality after several rounds of chemotherapy treatments before T cell isolation. For patients with T cell malignancies, it is difficult to isolate healthy T cells. Therefore, there is an unmet medical need to produce “off-the-shelf” universal CAR-T (UCAR-T) cells from healthy donors for allogeneic therapeutic use.

The present disclosure provides solutions to the unmet needs in the art of allogeneic off-the-shelf immunotherapy for the treatment of cancers. The present disclosure is directed to systems, compositions and methods to expand the use of engineered immune cells having receptors that target two or more targets.

Described herein are universal chimeric antigen receptor (UCAR)-expressing immune cells (e.g., lymphocytes, such as T cells (UCAR-T cells), and their use in treating diseases (e.g., cancer) and other physiological conditions. More specifically, described herein are UCAR-T cells and their use in treating diseases associated with CD33 and/or CD5 expression, such as acute myeloid leukemia (AML) and/or T cell malignancies. The UCAR-T cells contain a nucleic acid construct(s) encoding a chimeric antigen receptor(s) targeting CD5 and CD33 and are referred to herein as “dual-CAR T cells”.

An embodiment is a transgenic lymphocyte that expresses a CAR that includes a signal peptide, an extracellular domain including variable light V_{L} and a variable heavy V_{H} domains that bind CD5 and V_{L} and V_{H} domains that bind CD33, a hinge domain, a transmembrane domain and a costimulatory domain.

In some embodiments, the signal peptide is N-terminal to the V L domain that binds CD5, which is N-terminal to the V_{H} domain that binds CD33, which is N-terminal to the V_{L} domain that binds CD33, which is N-terminal to the V_{H} domain that binds CD5. In some embodiments, the signal peptide is N-terminal to the V_{H} domain that binds CD5, which is N-terminal to the V_{L} domain that binds CD33, which is N-terminal to the V_{H} domain that binds CD33, which is N-terminal to the V_{L} domain that binds CD5. In some embodiments, the signal peptide is N-terminal to the V_{L} domain that binds CD33, which is N-terminal to the V_{H} domain that binds CD5, which is N-terminal to the V_{L} domain that binds CD5, which is N-terminal to the V_{H} domain that binds CD33. In some embodiments, the signal peptide is N-terminal to the V_{H} domain that binds CD33, which is N-terminal to the V_{L} domain that binds CD5, which is N-terminal to the V_{H} domain that binds CD5, which is N-terminal to the V L domain that binds CD33. In some embodiments, the signal peptide is a CD8a signal peptide, a GM-CSF signal peptide, a CD4 signal peptide, a CD137 (4-1BB) signal peptide, or a combination thereof. In some embodiments, one or more of the linker domains is (G4S)n wherein n is 1 or 3, a 218 linker, or a combination thereof.

In some embodiments, the hinge domain is a CD8α hinge domain, a CD28 hinge domain, a CD137 hinge domain, an IgG1 hinge domain, an IgG2 hinge domain, an IgG3 hinge domain, an IgG4 hinge domain, or a combination thereof.

In some embodiments, the transmembrane domain is a CD8α transmembrane domain, a CD28 transmembrane domain, a CD3e transmembrane domain, a CD45 transmembrane domain, a CD4 transmembrane domain, a CD5 transmembrane domain, a CD9 transmembrane domain, a CD16 transmembrane domain, a CD22 transmembrane domain, a CD33 transmembrane domain, a CD37 transmembrane domain, a CD64 transmembrane domain, a CD80 transmembrane domain, a CD86 transmembrane domain, a CD134 transmembrane domain, a CD137 transmembrane domain, a transmembrane domain CD154, or a combination thereof.

In some embodiments, the costimulatory domain is a 4-1BB costimulatory domain, a CD28 costimulatory domain, a OX40 costimulatory domain, a CD2 costimulatory domain, a CD7 costimulatory domain, a CD27 costimulatory domain, a CD28 costimulatory domain, a CD30 costimulatory domain, a CD40 costimulatory domain, a CD70 costimulatory domain, a CD134 costimulatory domain, a PD1 costimulatory domain, an ICOS costimulatory domain, an NKG2D costimulatory domain, a GITR costimulatory domain, a TLR2 costimulatory domain, or a combination thereof.

The V_{H} domain that binds CD5 includes an amino acid sequence that is at least 95% identical to SEQ ID NO: 26 or SEQ ID NO: 28. The V_{L} domain that binds CD5 includes an amino acid sequence that is at least 95% identical to SEQ ID NO: 27 or SEQ ID NO: 29. The V_{H} domain that binds CD33 includes an amino acid sequence that is at least 95% identical to SEQ ID NO: 30. The V_{L} domain that binds CD33 includes an amino acid sequence that is at least 95% identical to SEQ ID NO: 31. In some embodiments, the CAR includes an amino acid sequence that is at least 95% identical to SEQ ID NO: 14. In some embodiments, the CAR includes an amino acid sequence that is at least 95% identical to SEQ ID NO: 15. In some embodiments, the CAR includes an amino acid sequence that is at least 95% identical to SEQ ID NO: 16. In some embodiments, the CAR includes an amino acid sequence that is at least 95% identical to SEQ ID NO: 17. In some embodiments, the CAR includes an amino acid sequence that is at least 95% identical to SEQ ID NO: 18. In some embodiments, the CAR includes an amino acid sequence that is at least 95% identical to SEQ ID NO: 19. In some embodiments, the CAR includes an amino acid sequence that is at least 95% identical to SEQ ID NO: 20. In some embodiments, the CAR includes an amino acid sequence that is at least 95% identical to SEQ ID NO: 21. In some embodiments, the CAR includes an amino acid sequence that is at least 95% identical to SEQ ID NO: 22. In some embodiments, the CAR includes an amino acid sequence that is at least 95% identical to SEQ ID NO: 23.

In some embodiments, the CAR includes an amino acid sequence selected from the group consisting of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23.

In some embodiments, the transgenic lymphocyte is a T cell. The T cell expresses T-cell receptor alpha chain or T-cell receptor beta chain at a level that does not elicit a graft-versus-host disease (GVHD) response when the transgenic lymphocyte is administered to a patient. In some embodiments, the transgenic lymphocyte is a natural killer cell. In some embodiments, the transgenic lymphocyte expresses an exogenous nucleic acid encoding a cytokine or a cytokine receptor gene. The encoded cytokine or cytokine receptor gene is interleukin 2, interleukin 7, interleukin 12, interleukin 15, or interleukin 21. In some embodiments, the lymphocyte expresses an exogenous nucleic acid encoding a suicide gene. In some embodiments, the pathological condition is a T-cell malignancy.

In an embodiment, a transgenic lymphocyte expresses a CAR that binds CD5 and a CAR that binds CD33. The CAR that binds CD5 includes a signal peptide, an extracellular domain including V_{L} and V_{H} domains that bind CD5, a hinge domain, a transmembrane domain and a costimulatory domain. The CAR that binds CD33 includes a signal peptide, an extracellular domain including V_{L} and V_{H} domains that bind CD33, a hinge domain, a transmembrane domain and a costimulatory domain.

In some embodiments, the CAR that binds CD33 the signal peptide is N-terminal to the V_{L} domain that binds CD33, which is N-terminal to the V_{H} domain that binds CD33, which is N-terminal to the hinge domain. In some embodiments, the CAR that binds CD5, the signal peptide is N-terminal to the V_{L} domain that binds CD5, which is N-terminal to the V_{H} domain that binds CD5, which is N-terminal to the hinge domain.

In some embodiments, the CAR that binds CD5 includes an amino acid sequence that is at least 95% identical to SEQ ID NO: 2. In some embodiments, the CAR that binds CD5 includes an amino acid sequence that is at least 95% identical to SEQ ID NO: 3. In some embodiments, the CAR that binds CD5 includes an amino acid sequence that is at least 95% identical to SEQ ID NO: 4. In some embodiments, the CAR that binds CD5 includes an amino acid sequence that is at least 95% identical to SEQ ID NO: 5. In some embodiments, the CAR that binds CD5 includes an amino acid sequence that is at least 95% identical to SEQ ID NO: 6. In some embodiments, the CAR that binds CD5 includes an amino acid sequence that is at least 95% identical to SEQ ID NO: 7. In some embodiments, the CAR that binds CD5 includes an amino acid sequence selected from the group consisting of SEQ ID NO: 2, SEQ ID NO: 3, SEQ ID NO: 4, SEQ ID NO: 5, SEQ ID NO: 6 and SEQ ID NO: 7.

In some embodiments, the CAR that binds CD33 includes an amino acid sequence that is at least 95% identical to SEQ ID NO: 8. In some embodiments, the CAR that binds CD33 includes an amino acid sequence that is at least 95% identical to SEQ ID NO: 9. In some embodiments, the CAR that binds CD33 includes an amino acid sequence that is at least 95% identical to SEQ ID NO: 10. In some embodiments, the CAR that binds CD33 includes an amino acid sequence that is at least 95% identical to SEQ ID NO: 11. In some embodiments, the CAR that binds CD33 includes an amino acid sequence that is at least 95% identical to SEQ ID NO: 12. In some embodiments, the CAR that binds CD33 includes an amino acid sequence that is at least 95% identical to SEQ ID NO: 13. In some embodiments, the CAR that binds CD33 includes an amino acid sequence selected from the group consisting of SEQ ID NO: 8, SEQ ID NO: 9, SEQ ID NO: 10, SEQ ID NO: 11, SEQ ID NO: 12 and SEQ ID NO: 13.

In an embodiment, a method of making a transgenic lymphocyte that expresses a chimeric antigen receptor that binds CD5 and CD33 includes introducing a nucleic acid into a lymphocyte. The nucleic acid that is introduced into a lymphocyte encodes a signal peptide, an extracellular domain including V_{L} and V_{H} domains that bind CD5 and V_{L} and V_{H} domains that bind CD33, a hinge domain, a transmembrane domain and a costimulatory domain. In some embodiments, introducing the nucleic acid into the lymphocyte includes electroporation, transformation, or transduction. In some embodiments, introducing the nucleic acid is a viral vector, a non-viral vector, or naked DNA. The viral vector is a lentivirus vector or an adeno-associated virus vector. In some embodiments, the nucleic acid is integrated into the lymphocyte genome. The nucleic acid is a randomly integrated in the lymphocyte genome. In some embodiments, the method is performed under conditions that allow expression of the CAR in the transgenic lymphocyte. In some embodiments, the cell expresses the CAR upon introduction of the nucleic acid.

In an embodiment, a method of making a transgenic lymphocyte that expresses a CAR that binds CD5 and a CAR that binds CD33, wherein the CAR that binds CD5 and the CAR that binds CD33 are linked by a self-cleaving protein, and wherein the method includes introducing a nucleic acid into a lymphocyte. The nucleic acid encodes a CAR that binds CD5 that includes a signal peptide, an extracellular domain including V_{L} and V_{H} domains that bind CD5, a hinge domain, a transmembrane domain and a costimulatory domain. The nucleic acid also encodes a CAR that binds CD33 that includes a signal peptide, extracellular domain including V_{L} and V_{H} domains that bind CD33, a hinge domain, a transmembrane domain and a costimulatory domain.

In some embodiments, the self-cleaving peptide is a 2A peptide. In some embodiments, introducing the nucleic acid into the lymphocyte includes electroporation, transformation, or transduction. In some embodiments, the nucleic acid is introduced into the lymphocyte with a viral vector, a non-viral vector or as naked DNA. The viral vector is a lentivirus or an adeno-associated virus vector. In some embodiments, the nucleic acid is integrated into the lymphocyte genome. The nucleic acid is randomly integrated in the lymphocyte genome. In some embodiments, the method is performed under conditions that allow for expression of the CAR in the transgenic lymphocyte. In some embodiments, the cell expresses the CAR upon introduction of the nucleic acid.

In an embodiment, a nucleic acid encoding a CAR includes a signal peptide, an extracellular domain, a hinge domain, a transmembrane domain and a costimulatory domain. The extracellular domain includes V_{L} and V_{H} domains that bind CD5, V_{L} and V_{H} domains that bind CD33, and a linker domain between adjacent V_{L} and V_{H} domains.

In an embodiment, a nucleic acid encodes a CAR that binds CD5 and a CAR that binds CD33. The CAR that binds CD5 includes a signal peptide, an extracellular domain, a hinge domain, a transmembrane and a costimulatory domain. The extracellular domain includes V_{L} and V_{H} domains that bind CD5 and a linker domain between adjacent V_{L} and V_{H} domains. The CAR that binds CD33 includes a signal peptide, an extracellular membrane, a hinge domain, a transmembrane domain and a costimulatory domain. The extracellular domain includes V_{L} and V_{H} domains that bind CD33 and a linker domain between adjacent V_{L} and V_{H} domains.

In an embodiment, a method of treating acute myeloid leukemia (AML) includes administering an effective amount of a transgenic lymphocyte to a patient in need thereof. In some embodiments, the transgenic lymphocyte is allogeneic. In some embodiments, the lymphocyte is autologous. In some embodiments, the AML includes leukemic cells that express CD33 as a cell-surface protein.

In an embodiment, a method of treating a T-cell malignancy includes administering a therapeutically effective amount of a transgenic lymphocyte to a patient in need thereof. In some embodiments, the transgenic lymphocyte is allogeneic. In some embodiments, the transgenic lymphocyte is autologous. In some embodiments, the T-cell malignancy includes T-cells that express CD5 as a cell-surface protein.

The transgenic lymphocytes described herein target CD5, which is associated with T cells of the patient. By targeting the T cells of the patient, the transgenic lymphocytes reduce host-versus-graft disease (HVGD). Targeting T cells of the patient (CD5) in combination with targeting cells associated with a disease state (CD33) provides synergistic benefits, as described further herein.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-B depict CD5 and CD33 expression on the surface of T-ALL, AML cell lines and genetically modified SK-Hep1 cell lines. Both T-ALL cell lines express 100% CD5 and both AML cell lines express CD33 (FIG. 1A). SK-Hep-1-CD5 and SK-Hep-1-CD33 cell lines were also generated by ectopically expressing CD5 and CD33 on SK-Hep-1 cells (FIG. 1B).

FIG. 2 depicts a TRAC gRNA screen that assesses TRAC knockout efficiency. T cells were activated by CD3/CD28 microbeads and electroporated with Crispr cas9 and indicated sgRNA with or without enhancer. TRAC KO efficiency is assessed by flow cytometry after staining of CD3 on day five after electroporation.

FIGS. 3A-B depict a CD5 gRNA screen that assesses CD5 knockout efficiency. T cells were activated by CD3/CD28 microbeads and electroporated with Crispr cas9 and indicated sgRNA. Five gRNAs were tested with and without the presence of an electroporation enhancer. CD5 knockout efficiency was assessed by flow cytometry after staining of CD5 on day five after electroporation.

FIGS. 4A-B depict CAR expression and purity of CD5 CARs on T cells. CD5 CAR expression were assessed with APC labeled CD5 antigen (FIG. 4A). T cell purity were assessed with anti-CD3 and anti-CD5 antibodies (FIG. 4B).

FIGS. 5A-B depict CAR5 CARs were further assessed and ranked by short-term cytotoxicity assay by mixing fresh CD5 CAR-T cells with CSFE-labeled allogeneic Pan-T cells (FIG. 5A) or CCRF-CEM-luc (FIG. 5B) at indicated ratio of Effector cells vs target cells. NC indicated no transduced T cells. CD19 CAR-T cells were used as a negative control.

FIGS. 6A-B depict different CD33 CARs (CAR33) expression on T cells (FIG. 6A) and CAR33 screen assessing CAR33 killing efficiencies on MOLM13-luc cells using luciferase based assay (FIG. 6B).

FIGS. 7A-F depict the layout of structure of CD5 and CD33 dual-CAR constructs. FIGS. 7A-D are schematic illustrations depicting two loop structures. FIGS. 7E-F are schematic illustrations depicting peptide 2A (P2A) structures.

FIG. 8 depicts single CAR and dual-CAR expression in various indicated constructs 11 days after transduction with the lentiviral vector constructs encoding either CAR5, CAR33 or dual CAR5-CAR33 with the corresponding scFv.

FIGS. 9A-B depict further assessment of in vitro function for single and dual-CAR constructs using real-time cellular impedance monitoring technology (RTCA) by mixing fresh CAR-T cells with CD5-SK-Hep-1 (FIG. 9A) or CD33-SK-Hep-1 (FIG. 9B) cells.

FIGS. 10A-C depict assessment function of dual-CAR constructs by in vitro cytotoxicity against AML and T-ALL cell lines using a luciferase-based assay. CAR-T cells were cocultured with MOLM13-luc (FIG. 10A), MV-4-11-luc (FIG. 10B) or CCRF-CEM-luc (FIG. 10C) at either 2:1 or 1:1 ratio (effector cells vs target cells) for 18 hours.

FIGS. 11A-D depict function of dual-CAR CAR-T cells cytotoxicity against autologous T cells or allogeneic T cells. Fresh dual-CAR T cells were cocultured with autologous T cells (FIGS. 11A-B) or allogeneic T cells (FIGS. 11C-D) at 2:1 or 1:1 ratio. Cytotoxicity were assessed at 18 hrs or 44 hrs after coculture by CFSE-based flow cytometry assay.

FIGS. 12A-F depict in vitro functional assessment of CD5/TCR knockout/IL15 armed dual CAR-T (CD5/CD33) cells. FIGS. 12A-B depict killing activity of CD5/TCR knockout/IL15 armed dual CAR-T (CD5/CD33) cell on MOLM13-luc (FIG. 12A) or allogeneic T cells (FIG. 12B) after coculturing for 18 h. FIGS. 12C-D depict propagation of CD5/TCR knockout dual CAR-T (CD5/CD33) cells when coculturing with MOLM13-luc cell line (FIG. 12C) or allogeneic T cells (FIG. 12D). FIGS. 12E-F depicts cytokine release (IFN-gamma) from CD5/TCR knockout dual CAR-T (CD5/CD33) cells upon stimulated by CD33 positive cells (FIG. 12E) or CD5 positive cells (FIG. 12F).

FIGS. 13A-G depict CD5/TCR knockout/IL15 armed dual CAR-T (CD5/CD33) cells (UCAR-T) avoid rejection from allogeneic PBMC. UCAR-T cells (HLA-A2+) were coculture with allogeneic PBMC (HLA-A2−), T cell depleted allogeneic PBMC (PBMC-T) or NK cell depleted allogeneic PBMC (PBMC-NK). UCAR-T and allogeneic T cells were gated out by CD3 and HLA-A2 (FIGS. 13A-C). FIGS. 13D-G depict the quantification of the HLA-A2+ numbers at each indicated day relative to the numbers at Day 0.

FIGS. 14A-C depict antitumor efficacy of CD5/TCR knockout/IL15 armed dual CAR-T (CD5/CD33) cells in MOLM13-luc and allogeneic T cells xenograft model. FIGS. 14A-B represent the bioluminescent imaging and signal of MOLM13-luc cancer growth. FIG. 14C depicts Kaplan-Meier curve generated from survival of animals in FIG. 14A.

FIGS. 15A-C depict flow cytometry analysis of cancer cells (HLA-A2−/Human CD3−), allogeneic T cells (HLA-A2−/Human CD3+) and CD5/TCR knockout/IL15 armed dual CAR-T (CD5/CD33) cells (HLA-A2 positive) in mice. Human cells were gated out with anti-mCD45 and anti-hCD45 (FIG. 15A). T cells were gated out with anti-hCD3 and anti-hHLA-A2. UCAR-T cells are HLA-A2 positive and allogenic T cells are HLA-A2 negative (FIG. 15B). CD33 CAR expression were assessed by stained with CD33 antigen (FIG. 15C).

Throughout the figures, “NC” is an abbreviation for “no CAR-T.”

### DETAILED DESCRIPTION

A description of example embodiments follows.

An allogeneic cell therapy strategy targeting both patients' immune T cells and cancer cells shows great efficacy in reducing cancer levels in those patients. In brief, described herein are the aforementioned dual-CAR system and T cells expressing dual-CAR, and methods of producing the dual-CAR system and the T cells expressing the dual-CAR. The dual-CAR system eliminates both CD5+ and CD33+ cells, including CD5/CD33 ectopically expressed cell lines, tumor cell lines that express CD5 or CD33, and Pan-T cells from both the same person and a different person. Thus, these CAR-T cells clear tumor cells expressing CD5 such as some T cell malignancies, and also CD33-expressing tumor cells as seen in, for example, AML. Since the dual-CAR constructs can eliminate allogeneic T cells, one use of them is for producing off-the-shelf universal CAR-T cells for allogeneic treatment of AML and other T cell malignancies. TRAC is knocked out by gene editing technologies so that the CAR-T cells will not cause graft-versus-host disease (GVHD) during allogeneic cell therapy. Since CAR5 eliminates endogenous T cells in patients, CAR-T cells avoid rejection by the patients' immune system (HVG), leading to prolonged survival and function. These dual-CAR modified T cells are expected to have strong tumor elimination efficacy in humans against both AML and other T cell malignancies. The anti-tumor function of the current system can also be further improved by combining it with cytokines including IL2, IL7, IL15, and IL21, the signaling of which has been demonstrated to significantly augment CAR-T cell persistence in vivo.

In some embodiments, the UCAR-T cells either do not express TCR α chain and/or β chain, or express a low level of TCR α chain and/or β chain (e.g., to avoid eliciting graft-versus-host disease (GVHD) during in vivo use). The UCAR-T cells can also be engineered so that they either do not express CD5 or express a low level of CD5 (e.g., to limit fratricide among UCAR-T cells in vivo). The UCAR-T can also contain a nucleic acid encoding a cytokine (for example, IL2, IL7, IL12, IL15, or IL21) or cytokine receptor (e.g., to increase persistence and expansion of UCAR-T cells in vivo). The UCAR-T cells can further contain a nucleic acid comprising a suicide gene (e.g., so that in the event of a strong side effect during treatment of a patient, the UCAR-T cells can be eliminated).

### Acute Myeloid Leukemia

As used herein, “Acute Myeloid Leukemia,” or AML, is a cancer of cells originating in the bone marrow and often moving quickly into the blood, as well. AML infects white blood cells (WBCs) referred to as “lymphocytes.” Lymphocytes are mature white blood cells that develop from lymphoblasts in the bone marrow. Lymphocytes are the main cells that make up lymph tissue, a major part of the immune system that is found in lymph nodes, the thymus, the spleen, the tonsils and adenoids. In most AML, cells express CD33 as a cell-surface protein.

For treating AML patients, and without wishing to be bound by theory, it is believed that dual-targeting CAR cells described herein are able to avoid rejection by recipient patients by targeting CD5 expressed on the surfaces of the patients' T cells, and are able to kill diseased cells which express CD33 as well as CD33-expressing hematopoietic stem cells and other myeloid cells in recipient patients by targeting CD33, thereby creating space for UCAR-T expansion in vivo.

### T-Cell Malignancies

As used herein, a “T-cell malignancy” is a lymphoma that affects T cells. T-cell malignancies are a heterogenous group of disorders of clonal growth and T-cell dysfunction broadly grouped into T-cell lymphomas (TCLs) and T-cell leukemias, with mature and precursor subtypes. Despite progress in T-cell malignancies, there remains a need for new, targeted regimens to improve outcomes, particularly for relapsed and refractory patients. In some T-cell malignancies, T-cells express CD5 as a cell-surface protein (e.g., CD5-positive hematopoietic malignancies).

For treating T cell malignancies, and without wishing to be bound by theory, it is believed that dual-targeting CAR cells described herein can kill CD5 and/or CD33 expressing T cell tumor cells, normal T cells and CD33-expressing hematopoietic stem cells. In some embodiments, the patient may also receive an allogeneic hematopoietic stem cell transplantation.

### Overview of Chimeric Antigen Receptors

A chimeric antigen receptor (CAR) is a protein that binds a specified antigen. The chimeric antigen receptors described herein are designed to bind to one or more of cluster differentiation 5 (CD5) and cluster differentiation 33 (CD33).

In some embodiments, a transgenic lymphocyte expresses a CAR that binds CD5 and a CAR that binds CD33. The CAR that binds CD5 includes a signal peptide, an extracellular domain including V_{L} and V_{H} domains that bind CD5, a hinge domain, a transmembrane domain and a costimulatory domain. The CAR that binds CD33 includes a signal peptide, an extracellular domain including V_{L} and V_{H} domains that bind CD33, a hinge domain, a transmembrane domain and a costimulatory domain. Linker domains can be included between adjacent domains.

Lymphocytes can be genetically modified to express chimeric antigen receptors. The genetically modified lymphocytes that express a CAR can be administered to patient for treatment of a disease, such as cancer. In this way, lymphocytes are altered so that they bind and attack cells associated with a disease. Two types of lymphocytes that can be genetically modified to express a chimeric antigen receptor are T cells and natural killer (NK) cells. These are referred to as CAR T cells and CAR NK cells, respectively.

CAR T cells can be created by collecting blood from a patient and introducing a gene for the CAR into the patient's T cells. To engineer CAR T cells, a patient's blood is drawn and collected. White blood cells, including T cells, are collected from a blood sample. The gene for the desired chimeric antigen receptor is transduced into the T cells in vitro. Those CAR T cells are multiplied and then transferred to the patient by infusion. The CAR T cells are then able to bind to the target antigen on the cancer cells in the patient.

### Dual-CAR T Cells

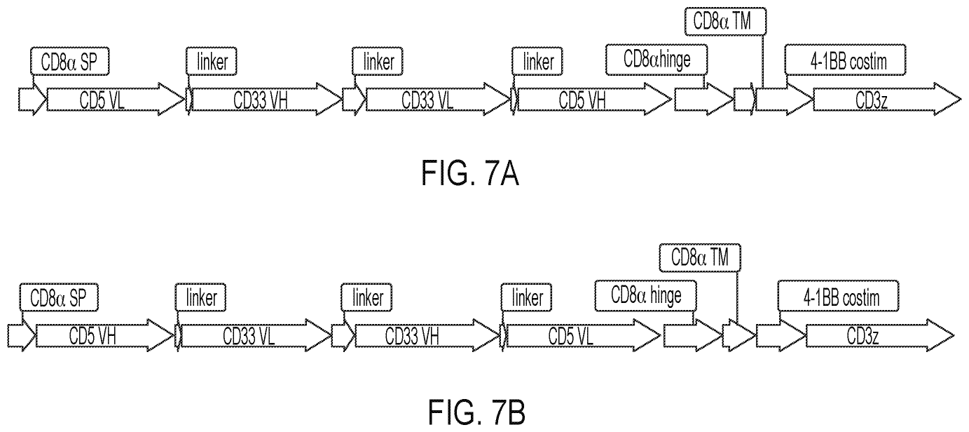

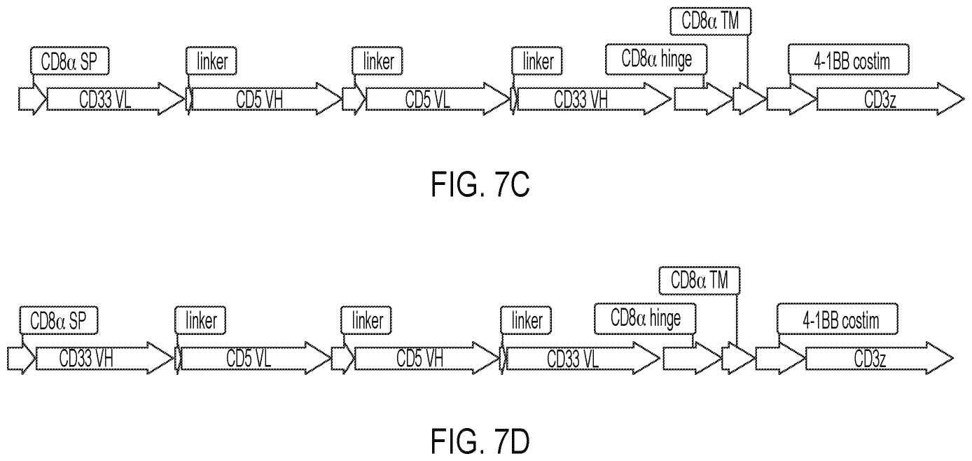

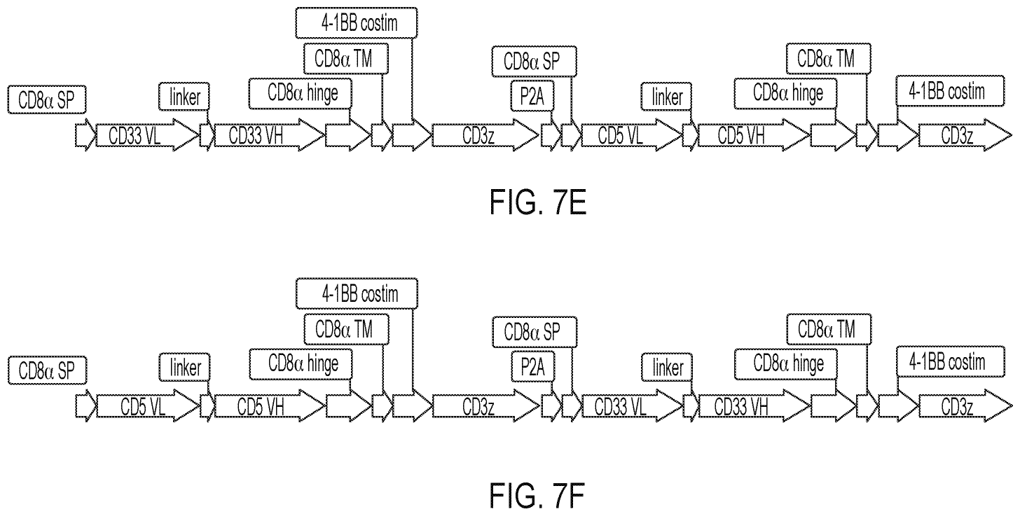

As used herein, the term “dual-CAR T cells” refers to CAR T cells engineered to express two tumor-associated antigen receptors on one cell at the same time, reducing the likelihood that T cells will attack non-tumor cells. FIGS. 7A-F illustrates multitarget CAR T cells configured in tandem and in loop. Tandem CARs contain two different scFvs in a single CAR molecule that can either be stacked in series or as a looped structure.

Expression of two CAR molecules in one viral plasmid may require codon optimization of duplicated DNA sequences to reduces chances of DNA recombination. An alternative is to design a receptor fusing two scFvs with different specificities with a single intracellular module in tandem. Tandem CARs have the advantage of smaller transgene size when compared with dual CARs, which is important when other transgenes (i.e., cytokines) are also incorporated into the CAR plasmid.

In some embodiments, a nucleic acid encoding a CAR includes a signal peptide, an extracellular domain, a hinge domain, a transmembrane domain and a costimulatory domain. The extracellular domain includes V_{L} and V_{H} domains that bind CD5, V_{L} and V_{H} domains that bind CD33, and a linker domain between adjacent V_{L} and V_{H} domains. Linker domains can be included between adjacent domains.

In some embodiments, a nucleic acid encodes a CAR that binds CD5 and a CAR that binds CD33. The CAR that binds CD5 includes a signal peptide, an extracellular domain, a hinge domain, a transmembrane and a costimulatory domain. The extracellular domain includes V_{L} and V_{H} domains that bind CD5 and a linker domain between adjacent V_{L} and V_{H} domains. The CAR that binds CD33 includes a signal peptide, an extracellular membrane, a hinge domain, a transmembrane domain and a costimulatory domain. The extracellular domain includes V_{L} and V_{H} domains that bind CD33 and a linker domain between adjacent V_{L} and V_{H} domains.

In some embodiments, CAR5 and CAR33 are expressed in the same construct and two scFvs are linked in a loop. In some embodiments, CAR5 and CAR33 are in the same construct and the two CARs are linked via a 2A self-cleaving peptide or IRES.

The dual-CAR can be constructed using single-chain variable fragment (scFv) and/or single-domain variable fragment (sdFv, V_{H} H, or nanobody) corresponding to CD5 and CD33 antigens in the same construct or in separate constructs.

### Signal Peptide Domain

A “signal peptide” refers to a peptide sequence present at the N-terminus of the CAR that directs the protein to the endoplasmic reticulum and, subsequently, the T cell surface. The signal peptide domain can be a full-length domain or a fragment thereof. In the CAR, the scFv may be fused to a TCR membrane and endodomain. A spacer may be included between the scFv and the TCR transmembrane to allow for variable orientation and antigen binding. The signal peptide directs the transport and localization of the protein within a cell, e.g. to a certain cell organelle (such as the endoplasmic reticulum) and/or the cell surface.

The signal peptide domain is usually N-terminal to the extracellular domain of the CAR, and typically is at the N-terminus of the CAR (i.e., the most N-terminal domain). In some embodiments, the signal peptide is a CD8α signal peptide (SEQ ID NO: 40), a granulocyte-macrophage colony-stimulating factor (GM-CSF) signal peptide (SEQ ID NO: 41), a CD4 signal peptide (SEQ ID NO: 42), a CD137 (4-1BB) signal peptide (SEQ ID NO: 43), or a combination thereof. In some embodiments, one or more of the linker domains is (G4S)n wherein n is 1 or 3, a 218 linker (SEQ ID NO: 44), or a combination thereof.

### Extracellular Domains

An “extracellular domain” refers to the antigen recognition domain of the chimeric antigen receptor exposed to the outside of the cell. The extracellular domain can be a full-length domain or a fragment thereof. The extracellular domain interacts with its target antigen and is responsible for targeting the CAR T cell to any cell expressing a matching molecule. The extracellular domain can be derived from the variable regions of a monoclonal antibody and can be, e.g., a single-chain variable fragment (scFv) of a monoclonal antibody. An scFv can include a variable light (V L) and a variable heavy (V_{H}) region, which can have a binding ability to a target antigen (e.g., CD33 or CD5). Other examples for the extracellular domain include a single-domain antibody (sdAb), such as a V_{H} H sdAb.

In some embodiments, the signal peptide is N-terminal to the V_{L} domain that binds CD5, which is N-terminal to the V_{H} domain that binds CD33, which is N-terminal to the V_{L} domain that binds CD33, which is N-terminal to the V_{H} domain that binds CD5. In some embodiments, the signal peptide is N-terminal to the V_{H} domain that binds CD5, which is N-terminal to the V_{L} domain that binds CD33, which is N-terminal to the V_{H} domain that binds CD33, which is N-terminal to the V_{L} domain that binds CD5. In some embodiments, the signal peptide is N-terminal to the V_{L} domain that binds CD33, which is N-terminal to the V_{H} domain that binds CD5, which is N-terminal to the V_{L} domain that binds CD5, which is N-terminal to the V_{H} domain that binds CD33. In some embodiments, the signal peptide is N-terminal to the V_{H} domain that binds CD33, which is N-terminal to the V_{L} domain that binds CD5, which is N-terminal to the V_{H} domain that binds CD5, which is N-terminal to the V L domain that binds CD33.

CARs are not confined to using scFvs as the targeting ectodomain and other ligands and receptors have been substituted. For example, interleukin-specific CARs have been prepared by modifying interleukin molecules to form ectodomains. Cytokines, innate immune receptors, tumor necrosis factor receptors, growth factors, and structural proteins can be used as CAR extracellular domains.

Suitable CD5 CAR scFv include H65, hH65, and others that are commercially available and/or described in literature. Humanized versions can also be used.

Suitable CD33 CAR scFv include My9.6, humanized My9.6, M195, lintuzumab (HuM195), and others that are commercially available and/or described in literature. Humanized versions can also be used.

In some embodiments, the extracellular domain of the CD5 CAR includes V_{L} and V_{H} domains that bind CD5 and a linker domain between adjacent V_{L} and V_{H} domains. In some embodiments, the extracellular domain of the CD33 CAR includes V_{L} and V_{H} domains that bind CD33 and a linker domain between adjacent V_{L} and V_{H} domains.

### Hinge Domains

As used herein, the term “hinge domain” refers to an extracellular structural region of the CAR that separates the binding units from the transmembrane domain. The hinge domain can be a full-length domain or a fragment thereof. The majority of CARs are designed with immunoglobulin (Ig)-like domain hinges. The hinge domain is also referred to as a “spacer.” These spacers generally supply stability for efficient CAR expression and activity. The hinge domain provides flexibility to access the target antigen. Hinge domains can also affect the overall performance of the CAR T cells.

In some embodiments, the hinge domain is a CD8α hinge domain, a CD28 hinge domain, a CD137 hinge domain, an IgG1 hinge domain, an IgG2 hinge domain, an IgG3 hinge domain, an IgG4 hinge domain, or a combination thereof.

### Transmembrane Domain

As used herein, the term “transmembrane domain” refers to an amino acid sequence that spans the cell membrane. The transmembrane domain can be a full-length domain or a fragment thereof. Typically, the transmembrane domain is a hydrophobic domain. Often, the transmembrane domain is an alpha helix. Although the main function of the transmembrane is to anchor the CAR in the T cell membrane, the transmembrane domain can be relevant to CAR T cell function.

In some embodiments, the transmembrane domain is a CD8α transmembrane domain, a CD28 transmembrane domain, a CD3e transmembrane domain, a CD45 transmembrane domain, a CD4 transmembrane domain, a CD5 transmembrane domain, a CD9 transmembrane domain, a CD16 transmembrane domain, a CD22 transmembrane domain, a CD33 transmembrane domain, a CD37 transmembrane domain, a CD64 transmembrane domain, a CD80 transmembrane domain, a CD86 transmembrane domain, a CD134 transmembrane domain, a CD137 transmembrane domain, a transmembrane domain CD154, or a combination thereof.

### Costimulatory Domain

As used herein, the term “costimulatory domain” refers to a region that can enhance antigen-specific cytotoxicity and/or cytokine production of a CAR T cell. The costimulatory domain can be a full-length domain or a fragment thereof. Intracellular domains are usually derived from costimulatory molecules. Costimulatory signals contribute to improved CAR T cell expansion, function, persistence and antitumor activity. These can be provided by incorporating intracellular signaling domains from one or more T cell costimulatory molecules.

In some embodiments, the costimulatory domain is a 4-1BB costimulatory domain, a CD28 costimulatory domain, a OX40 costimulatory domain, a CD2 costimulatory domain, a CD7 costimulatory domain, a CD27 costimulatory domain, a CD28 costimulatory domain, a CD30 costimulatory domain, a CD40 costimulatory domain, a CD70 costimulatory domain, a CD134 costimulatory domain, a PD1 costimulatory domain, an ICOS costimulatory domain, an NKG2D costimulatory domain, a GITR costimulatory domain, a TLR2 costimulatory domain, or a combination thereof.

### Linker Domain

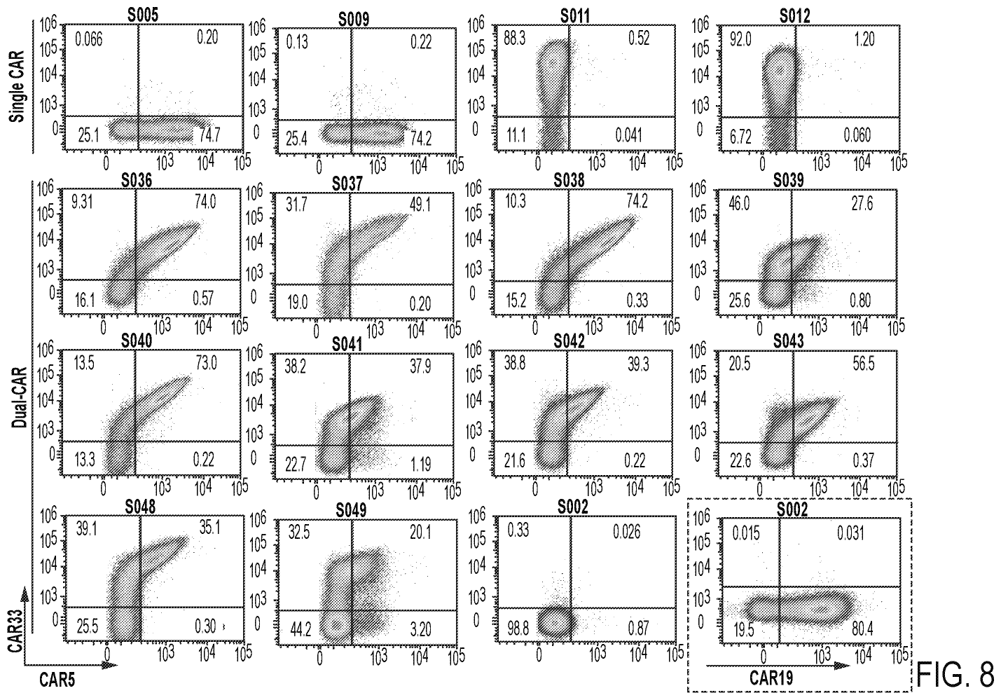

A “linker domain” refers to peptide segments covalently linking two adjacent domains within a protein. The linker domain can be a full-length domain or a fragment thereof. As illustrated in FIGS. 8A-F, the linker domain connects the target-specific extracellular domain and the transmembrane domain. Linker domains can also connect adjacent V_{L} and V_{H} domains. Linker domains play a variety of structural and functional roles in naturally occurring proteins. Linker domains have a role, for example, in tuning biological activities of the connected domains, in allosteric coupling and in viral replication. Linker domains are relevant in protein engineering, for example the alternation of functionality of engineered antibodies.

In some embodiments, one or more of the linker domains is (G4S)n wherein n is 1 or 3, a 218 linker (SEQ ID NO: 44), or a combination thereof.

### Nucleic Acids

A “nucleic acid” refers to a polymer including multiple nucleotide monomers (e.g., ribonucleotide monomers or deoxyribonucleotide monomers). “Nucleic acid” includes, for example, DNA (e.g., genomic DNA and cDNA), RNA, and DNA-RNA hybrid molecules. Nucleic acid molecules can be naturally occurring, recombinant, or synthetic. In addition, nucleic acid molecules can be single-stranded, double-stranded or triple-stranded. In certain embodiments, nucleic acid molecules can be modified. In the case of a double-stranded polymer, “nucleic acid” can refer to either or both strands of the molecule.

A “nucleotide” and a “nucleotide monomer” refer to naturally occurring ribonucleotide or deoxyribonucleotide monomers, as well as non-naturally occurring derivatives and analogs thereof. Accordingly, nucleotides can include, for example, nucleotides including naturally occurring bases (e.g., adenosine, thymidine, guanosine, cytidine, uridine, inosine, deoxyadenosine, deoxythymidine, deoxyguanosine, or deoxycytidine) and nucleotides including modified bases known in the art.

A “sequence identity,” refers to the extent to which two nucleotide sequences, or two amino acid sequences, have the same residues at the same positions when the sequences are aligned to achieve a maximal level of identity, expressed as a percentage. For sequence alignment and comparison, typically one sequence is designated as a reference sequence, to which a test sequences are compared. The sequence identity between reference and test sequences is expressed as the percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide or amino acid residue at 70% of the same positions over the entire length of the reference sequence.

Alignment of sequences for comparison to achieve maximal levels of identity can be readily performed by a person of ordinary skill in the art using an appropriate alignment method or algorithm. In some instances, the alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Natl. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology).

When using a sequence comparison algorithm, test and reference sequences are input into a computer, subsequent coordinates are designated, if necessary, and sequence algorithm program parameters are designated. The sequence comparison algorithm then calculates the percent sequence identity for the test sequence(s) relative to the reference sequence, based on the designated program parameters. A commonly used tool for determining percent sequence identity is Protein Basic Local Alignment Search Tool (BLASTP) available through National Center for Biotechnology Information, National Library of Medicine, of the United States National Institutes of Health. (Altschul et al., J. Mol Biol. 215(3):403-10 (1990)).

In various embodiments, two nucleotide sequences, or two amino acid sequences, can have at least, e.g., 70%, 75%, 80%, 85%, 86%, 87%, 88%, 89%, 90%, 91%, 92%, 93%, 94%, 95%, 96%, 97%, 98%, 99%, or more, sequence identity. When ascertaining percent sequence identity to one or more sequences described herein, the sequences described herein are the reference sequences.

In some embodiments, the variable light chain domain has at least 70%, 75%, 80%, 85%, 86%, 87%, 88%, 89%, 90%, 91%, 92%, 93%, 94%, 95%, 96%, 97%, 98%, 99%, or more, sequence identity to SEQ ID NO: 4. In some embodiments, the variable heavy chain domain has at least 70%, 75%, 80%, 85%, 86%, 87%, 88%, 89%, 90%, 91%, 92%, 93%, 94%, 95%, 96%, 97%, 98%, 99%, or more, sequence identity to SEQ ID NO: 8.

### Vectors

The terms “vector”, “vector construct” and “expression vector” mean the vehicle by which a DNA or RNA sequence (e.g. a foreign gene) can be introduced into a host cell, so as to transform the host and promote expression (e.g. transcription and translation) of the introduced sequence. Vectors typically include the DNA of a transmissible agent, into which foreign DNA encoding a protein is inserted by restriction enzyme technology. A common type of vector is a “plasmid”, which generally is a self-contained molecule of double-stranded DNA that can readily accept additional (foreign) DNA and which can be readily introduced into a suitable host cell. A large number of vectors, including plasmid and fungal vectors, have been described for replication and/or expression in a variety of eukaryotic and prokaryotic hosts.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. A polynucleotide or polypeptide is expressed recombinantly, for example, when it is expressed or produced in a foreign host cell under the control of a foreign or native promoter, or in a native host cell under the control of a foreign promoter.

Gene delivery vectors generally include a transgene (e.g., nucleic acid encoding an enzyme) operably linked to a promoter and other nucleic acid elements required for expression of the transgene in the host cells into which the vector is introduced. Suitable promoters for gene expression and delivery constructs are known in the art. Recombinant plasmids can also include inducible, or regulatable, promoters for expression of an enzyme in cells.

Various gene delivery vehicles are known in the art and include both viral and non-viral (e.g., naked DNA, plasmid) vectors. Viral vectors suitable for gene delivery are known to those skilled in the art. Such viral vectors include, e.g., vector derived from the herpes virus, baculovirus vector, lentiviral vector, retroviral vector, adenoviral vector, adeno-associated viral vector (AAV), and murine stem cell virus (MSCV). The viral vector can be replicating or non-replicating. Such vectors may be introduced into many appropriate host cells, using methods disclosed or cited herein or otherwise known to those skilled in the relevant art.

Non-viral vectors for gene delivery include naked DNA, plasmids, transposons, and mRNA, among others. Non-limiting examples include pKK plasmids (Clonetech), pUC plasmids, pET plasmids (Novagen, Inc., Madison, Wis.), pRSET or pREP plasmids (Invitrogen, San Diego, Calif), pMAL plasmids (New England Biolabs, Beverly, Mass.). Such vectors may be introduced into many appropriate host cells, using methods disclosed or cited herein or otherwise known to those skilled in the relevant art.

In some embodiments, the vector includes an internal ribosome entry site (IRES). In some embodiments, the vector includes a selection marker, such as an ampicillin resistance gene (Amp). In some embodiments, the nucleic acid encodes a fluorescent protein, such as green fluorescent protein (GFP). In some embodiments, the nucleic acid is suitable for subcloning into pMSCV-IRES-GFP between EcoRI and XhoI. In some embodiments, the vector contains a multiple cloning site (MCS) for the insertion of the desired gene.

Although the genetic code is degenerate in that most amino acids are represented by multiple codons (called “synonyms” or “synonymous” codons), it is understood in the art that codon usage by particular organisms is nonrandom and biased towards particular codon triplets. Accordingly, in some embodiments, the vector includes a nucleotide sequence that has been optimized for expression in a particular type of host cell (e.g., through codon optimization). Codon optimization refers to a process in which a polynucleotide encoding a protein of interest is modified to replace particular codons in that polynucleotide with codons that encode the same amino acid(s), but are more commonly used/recognized in the host cell in which the nucleic acid is being expressed. In some aspects, the polynucleotides described herein are codon optimized for expression in T cells.

### Methods of Making Transgenic Host Cells

Described herein are methods of making a transgenic host cell, such as transgenic T cells. The transgenic host cells can be made, for example, by introducing one or more of the vector embodiments described herein into the host cell. The transgenic host cell can be constructed by harvesting host cells from the host's blood, and then modifying the host cell to express a transgene encoding a tumor-specific CAR. Transgenes are introduced into the host cell genome using a vector carrying the transgene, as described herein. The transgenic host cells are then administered to the patient in need thereof.

The dual-CAR can be constructed using single-chain variable fragment (scFv) and/or single-domain variable fragment (sdFv, VHH or nanobody) corresponding to CD5 and CD33 antigens in the same construct or in separate constructs.

The method includes introducing into a host cell a vector that includes a nucleic acid that encodes for signal peptide, an extracellular domain including V_{L} and V_{H} domains that bind CD5 and V_{L} and V_{H} domains that bind CD33, a hinge domain, a transmembrane domain and a costimulatory domain. In some embodiments, introducing the nucleic acid into the lymphocyte includes electroporation, transformation, or transduction. In some embodiments, introducing the nucleic acid is a viral vector, a non-viral vector, or naked DNA. The viral vector is a lentivirus vector or an adeno-associated virus vector. In some embodiments, the nucleic acid is integrated into the lymphocyte genome. The nucleic acid is a randomly integrated in the lymphocyte genome. In some embodiments, the method is performed under conditions that allow expression of the CAR in the transgenic lymphocyte. In some embodiments, the cell expresses the CAR upon introduction of the nucleic acid.

In some embodiments, the nucleic acid encodes a CAR that binds CD5 and a CAR that binds CD33, wherein the CAR that binds CD5 and the CAR that binds CD33 are linked by a self-cleaving protein. The nucleic acid being introduced encodes a CAR that binds CD5 that includes a signal peptide, an extracellular domain including V_{L} and V_{H} domains that bind CD5, a hinge domain, a transmembrane domain and a costimulatory domain. The nucleic acid being introduced also encodes a CAR that binds CD33 that includes a signal peptide, extracellular domain including V_{L} and V_{H} domains that bind CD33, a hinge domain, a transmembrane domain and a costimulatory domain.

In some embodiments, introducing the nucleic acid into the lymphocyte includes electroporation, transformation, or transduction. In some embodiments, the nucleic acid is introduced into the lymphocyte with a viral vector, a non-viral vector or as naked DNA. The viral vector is a lentivirus or an adeno-associated virus vector. In some embodiments, the nucleic acid is integrated into the lymphocyte genome. The nucleic acid is randomly integrated in the lymphocyte genome. In some embodiments, the method is performed under conditions that allow for expression of the CAR in the transgenic lymphocyte. In some embodiments, the cell expresses the CAR upon introduction of the nucleic acid.

### αβ T-Cell Receptor Knockout

“αβ T-cell receptor (TCR) knockout” refers to the method that can be used to prevent host-versus-graft disease (HVGD) in a patient being administered CAR T cells. Endogenous αβ TCRs on infused allogeneic T cells may recognize major and minor histocompatibility antigens in the recipient leading to HVGD. To avoid HVGD during allogeneic CAR-T therapy using αβ T cells, αβ TCR can be knocked out using CRISPR/Cas9 technology, or other gene editing technologies known to persons skilled in the art. CD5 expression in UCAR-T cells can be deleted (e.g., knocked-out) to avoid fratricide from CD5 CARs. CRISPR/Cas9 knockout of endogenous αβ TCRs can increase the expression and functional activity of transduced CAR T cells.

### Methods of Treating Diseases

The dual-CAR T cells described herein can be used in methods of treating diseases in a subject. The dual-CAR T cells are administered to a subject (e.g., a patient) in need thereof.

Diseases that can be treated by administering the dual-CAR T cells disclosed herein include, but are not limited to, acute myeloid leukemia (AML) and T-cell malignancies. Typically, patients in need thereof have cells expressing the antigens CD33 and CD5. The dual-CAR T cells bind to cells expressing CD33 and CD5 antigens. For treating patients in need thereof, CD5 CAR removes patients' T cells so that CAR-T cells are not rejected by the patients' T cells and CD33 CAR kills patients' diseased cells which express CD33. CD33 CAR also kills CD33 expressing hematopoietic stem cells and other myeloid cells, creating space for CAR-T cell expansion. For treating T-cell malignancies, dual-CAR kills CD5 and/or CD33 expressing T cell tumors, followed by allogeneic hematopoietic stem cell transplantation.

In an embodiment, a method of treating AML includes administering an effective amount of a transgenic lymphocyte to a patient in need thereof. In some embodiments, the transgenic lymphocyte is allogeneic. In some embodiments, the lymphocyte is autologous. In some embodiments, the AML includes leukemic cells that express CD33 as a cell-surface protein.

In an embodiment, a method of treating a T-cell malignancy includes administering a therapeutically effective amount of a transgenic lymphocyte to a patient in need thereof. In some embodiments, the transgenic lymphocyte is allogeneic. In some embodiments, the transgenic lymphocyte is autologous. In some embodiments, the T-cell malignancy includes T-cells that express CD5 as a cell-surface protein

In some embodiments, dual-CART cells are administered at a dose of about 1 million IU/m² or less (e.g., about 800,000 IU/m²; 600,000 IU/m²; 400,000 IU/m²; 200,000 IU/m²; 100,000 IU/m²; 80,000 IU/m²; 60,000 IU/m²; 40,000 IU/m²; 20,000 IU/m²; 10,000 IU/m²; 8,000 IU/m²; 6,000 IU/m²; 4,000 IU/m²; 2,000 IU/m²; 1,000 IU/m²; 800 IU/m²; 600 IU/m²; 400 IU/m²; 200 IU/m²; 100 IU/m²). In some embodiments, dual-CAR T cells are administered at a dose greater than about 1 million IU/m² (e.g., 1 million IU/m² to about 5 million IU/m²).

Methods of introduction of dual-CAR T cells include, but are not limited to, intradermal, intramuscular, intraperitoneal, intravenous, subcutaneous, topical, oral and intranasal. Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectable.

As used herein, the term “subject” means an animal, preferably a mammal, e.g., a human, or a veterinary or agricultural animal such as a dog, cat, horse, cow, pig, sheep, goat, and the like.

As used herein, the terms “effective amount,” “therapeutically effective amount,” and a “sufficient amount” of a composition described herein refer to a quantity sufficient to, when administered to a subject, including a mammal (e.g., a human), effect beneficial or desired results, including effects at the cellular level, tissue level, or clinical results, and, as such, an “effective amount” or synonym thereto depends upon the context in which it is being applied. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art.

As used herein, “treatment” and “treating” refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those at risk to have the condition or disorder or those in which the condition or disorder is to be prevented.

### Values and Ranges

Unless otherwise indicated or otherwise evident from the context and understanding of one of ordinary skill in the art, values that are expressed as ranges can assume any specific value or subrange within the stated ranges in various embodiments, unless the context clearly dictates otherwise. “About” in reference to a numerical value generally refers to a range of values that fall within ±8%, in some embodiments ±6%, in some embodiments ±4%, in some embodiments ±2%, in some embodiments ±1%, in some embodiments ±0.5% of the value unless otherwise stated or otherwise evident from the context.

### EXEMPLIFICATION

The following examples refer to the constructs of Tables 1 and 2.

TABLE 1

| Construct | | |
|---|---|---|
| ID | Gene | SEQ ID |
| S002 | CAR19 | SEQ ID NO: 1 |
| S005 | CAR5-1 | SEQ ID NO: 2 |
| S006 | CAR5-2 | SEQ ID NO: 3 |
| S007 | CAR5-3 | SEQ ID NO: 4 |
| S008 | CAR5-4 | SEQ ID NO: 5 |
| S009 | CAR5-5 | SEQ ID NO: 6 |
| S010 | CAR5-6 | SEQ ID NO: 7 |
| S011 | CAR33-1 | SEQ ID NO: 8 |
| S012 | CAR33-2 | SEQ ID NO: 9 |
| S013 | CAR33-3 | SEQ ID NO: 10 |
| S014 | CAR33-4 | SEQ ID NO: 11 |
| S015 | CAR33-5 | SEQ ID NO: 12 |
| S016 | CAR33-6 | SEQ ID NO: 13 |
| S036 | Dual-CAR | SEQ ID NO: 14 |
| S037 | Dual-CAR | SEQ ID NO: 15 |
| S038 | Dual-CAR | SEQ ID NO: 16 |
| S039 | Dual-CAR | SEQ ID NO: 17 |
| S040 | Dual-CAR | SEQ ID NO: 18 |
| S041 | Dual-CAR | SEQ ID NO: 19 |
| S042 | Dual-CAR | SEQ ID NO: 20 |
| S043 | Dual-CAR | SEQ ID NO: 21 |
| S048 | Dual-CAR | SEQ ID NO: 22 |
| S049 | Dual-CAR | SEQ ID NO: 23 |
| S026 | CD5-GFP | SEQ ID NO: 24 |
| S027 | CD33-GFP | SEQ ID NO: 25 |
| | CD5 ScFV V_{H} in S036-S039, S048 | SEQ ID NO: 26 |
| | CD5 ScFV V_{L} in S036-S039, S048 | SEQ ID NO: 27 |
| | CD5 ScFV V_{H} in S040-S043, S049 | SEQ ID NO: 28 |
| | CD5 ScFV V_{L} in in S040-S043, | SEQ ID NO: 29 |
| | S049 | |
| | CD33 ScFV V_{H} in S036-S043, | SEQ ID NO: 30 |
| | S048-S049 | |
| | CD33 ScFV V_{L} in S036-S043, | SEQ ID NO: 31 |
| | S048-S049 | |

TABLE 2

| gRNA ID | SEQ ID |
|---|---|
| TRAC-gRNA1 | 32 |
| TRAC-gRNA2 | 33 |
| TRAC-gRNA3 | 34 |
| CD5-gRNA1 | 35 |
| CD5-gRNA2 | 36 |
| CD5-gRNA3 | 37 |
| CD5-gRNA4 | 38 |
| CD5-gRNA5 | 39 |

The sequences of SEQ ID NOs: 2-23 include a signal peptide domain, an extracellular domain comprising variable light (V_{L}) and variable heavy (V_{H}) domains, a hinge domain, a transmembrane domain, and a co-stimluatory domain.

### Pan-T Cell Isolation, Activation and Expansion

Leukopaks from healthy peripheral bloods were purchased from HemaCare. Pan T cells are isolated using EasySep Human T Cell Isolation Kit from Stemcell Technologies and frozen in CryoStor CS5 in a liquid N2 freezer. To use the cells, thaw and count, and resuspend the cells into X-VIVO 15 (Lonza) containing 5% FBS (Gibco) and 25 ng/ml IL2 (Peprotech). To activate Pan T cells, Dynabeads Human T-Activator CD3/CD28 (Thermo Fisher) are used. The cells are cultured in a 5% CO₂ incubator at 37° C. The Dynabeads are removed 2-3 days later, and the activated T cells are further cultured in X-VIVO 15+5% FBS+25 ng/ml IL2 for expansion.

### Cell Line and Culture

Cell line information as follows: 293T cells (ATCC, #CRL3216); human hepatic adenocarcinoma cell line SK-Hep-1 (ATCC, #HTB-52); acute T lymphocytic leukemia (T-ALL) Jurkat (ATCC, #TIB-152), and CCRF-CEM (ATCC, #CCL-119); acute myeloid leukemia (AML) MOLM13 (AddexBio, #C0003003); and MV-4-11 (ATCC, #CRL-9591). Subclones of SK-Hep-1 were generated by stably transducing cells with lentiviral vector encoding either CD5 or CD33. SK-Hep1-CD5 and SK-Hep1-CD33 cell lines were grown in RPMI medium supplemented with 10% heat-inactivated fetal bovine serum (FBS). Luciferase-expressing subclones in CCRF-CEM, MOLM13, and MV-4-11 were generated by stably transducing cells with lentiviral vector encoding firefly luciferase with GFP (Biosettia, GlowCell-16). CCRF-CEM-luciferase (luc), MOLM13-luc, MV-4-11-luc cell lines were cultured in EMEM medium supplemented with 10% heat-inactivated FBS. All the cells were switched to grow in RPMI 1640 medium with 10% FBS during cell cytolytic assay (killing assay). All cells were maintained at 37° C. in a humidified incubator with 5% CO₂.

### TRAC and CD5 Knockout Using CRISPR/Cas9 RNP

αβ TCR is knocked out using CRISPR/Cas9 technology. CD5 expression in UCAR-T cells is deleted to avoid fratricide from CD5 CARs. Three gRNAs are designed for human TRAC gene and five gRNAs for human CD5 gene based on IDT and Synthego online tools (high editing efficiency and less off-target editing potential) and gRNAs and HiFi Cas9 were ordered from IDT. Cas9 are produced and gRNA ribonucleoproteins (RNPs) are formed by incubating gRNA with Cas9 at 2.5:1 molar ratio at R.T. for 15 minutes. To screen for efficient gRNAs, Pan-T cells are activated using Dynabeads Human T-Activator CD3/CD28 (Thermo Fisher) for 2 days and RNPs are electroporated in the presence of electroporation enhancer (IDT) using Lonza 4D Nucleofector (Lonza Bioscience) using program EO-115. Knockout efficiencies are assessed using Attune NxT flow cytometer (Thermo Fisher) after staining for CD3 and/or CD5.

### Generation and Transduction of Recombinant Lentiviral Vectors Expressing CARs

293T cells are plated a day before the transfection. When cell confluence reached 75%, viral packaging vectors and transfer vector are cotransfected using the TransIT-VirusGEN® Transfection Reagent (Minis) and following the recommended protocol. Viral supernatant is collected 48 hours after the transfection, and the virus is further used for transduction. Pan-T cells from a second healthy donor are used to generate the chimeric antigen receptor (CAR) T cells. Pan-T cells are activated for two days with Dynabeads® Human T-Activator CD3/CD28 (Thermo Fisher Scientific), supplemented with 25 ng/ml of IL-2 two days prior to the transduction. Magnetic beads are removed from the T cells and the cells are electroporated with RNPs and infected with lentiviral vector encoding CAR constructs including CD5 CAR (CAR5), CD33 CAR (CAR33), and CD19 CAR (CAR19). T cells are expanded in X-VIVO medium supplement with 25 ng/ml of IL-2 followed by CAR expression and functional assays.

### Flow Cytometry

CD3 and CD5 are stained with a CD3-FITC antibody (BD Biosciences, #555339) and a CD5-APC antibody (BD Biosciences, #555355) for 30 minutes at R.T. in the dark. CAR5 surface expression is detected by staining with Human CD5 Protein, HisTag (AcroBiosystems, Cat #CD5-H52H5) for 1 hour at R.T., washed and then incubated with His-APC antibody (R&D Systems, IC050A) for 30 minutes at R.T. in the dark. CAR33 expression is stained with CD33 human protein-PE (Sino Biological, 12238-HCCH_P) for 1 hour at R.T. All the staining is performed at 1:10 dilutions. DAPI (Thermo Scientific, Cat #BV-421, 1:5000) is used to stain dead cells before running by the Attune NxT Flow Cytometer (Thermo Fisher Scientific). Data is further analyzed by either using Attune NxT flow software or FlowJo software.

### In Vitro Cell Cytotoxicity Assay (Killing Assay)

To assess cytotoxic function of CAR-T cells, various methods are used. For CF SE-flow based assay, Pan-T cells, CCRF-CEM, MOLM13, MV-4-11 cells are labeled with carboxyfluorescein succinimidyl ester (CFSE) (Biolegend, #423801) before co-incubated with CAR-T cells. Then, the number of live target cells are analyzed by flow cytometry after incubation for certain time (n=3). For luciferase-based assay, CCRF-CEM-luc, MOLM13-luc, and MV-4-11-luc cells are co-incubated with CAR-T cells at various E:T ratios for certain period and luminescence is read by a plate reader as an indicator of luciferase activity (n=3). For RTCA assay, SK-Hep-1-CD5 and SK-Hep-1-CD33 cells are plated one day before adding CAR-T cells at various effector to target (E:T) ratios. The cell cytotoxicity is measured by xCELLigence Real-Time Cell Analyzer (RTCA) (n=3). In all assays, the percentage of lysis is calculated using the mean of target only wells as no lysis activity and using the formula % lysis=100×(mean of the target only value−experimental group value)/(mean of the target only value).

### Cell Line Characterization and Generation

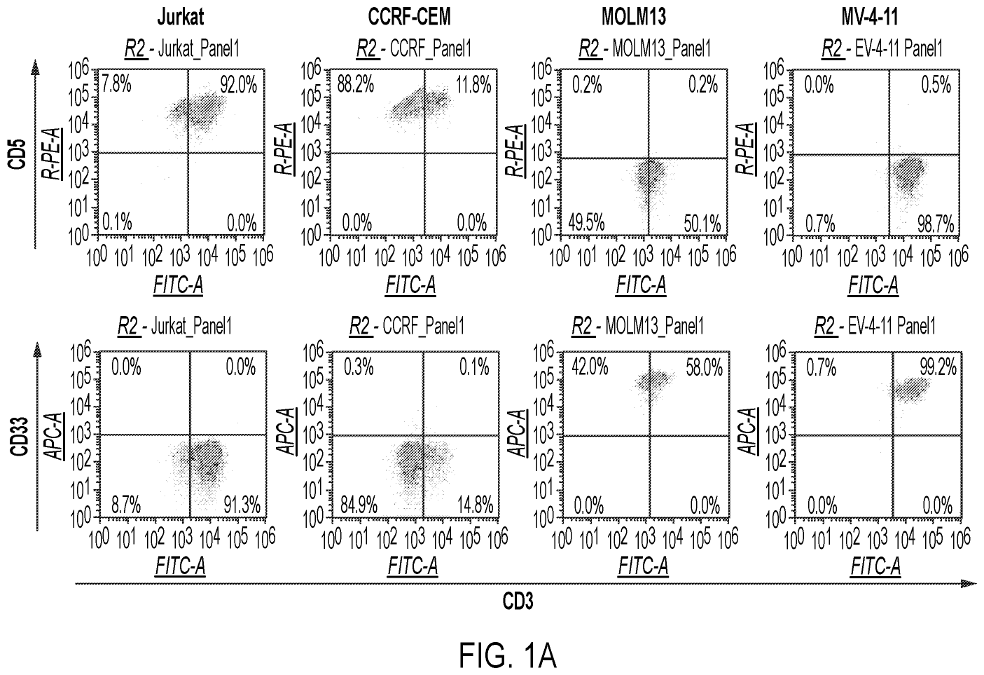

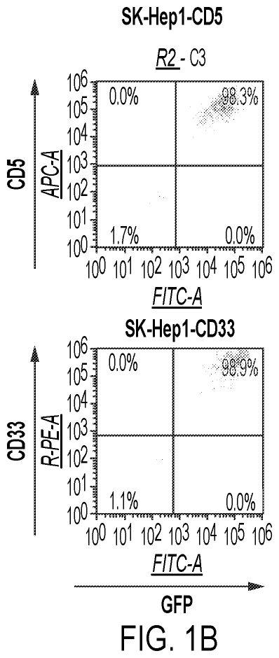

Expression of CD3, CD5 and CD33 on Jurkat, CCRF-CEM, MOLM13 and MV-4-11 is determined. As shown in FIG. 1A, both T-ALL cell lines (Jurkat and CCRF-CEM) express 100% CD5, and both AML (MOLM13 and MV-4-11) express CD33. SK-Hep1-CD5 and SK-Hep1-CD33 cell lines are also generated by ectopically expressing CD5 and CD33 on SK-Hep-1 cells (FIG. 1B).

TRAC Knockout gRNA Screen

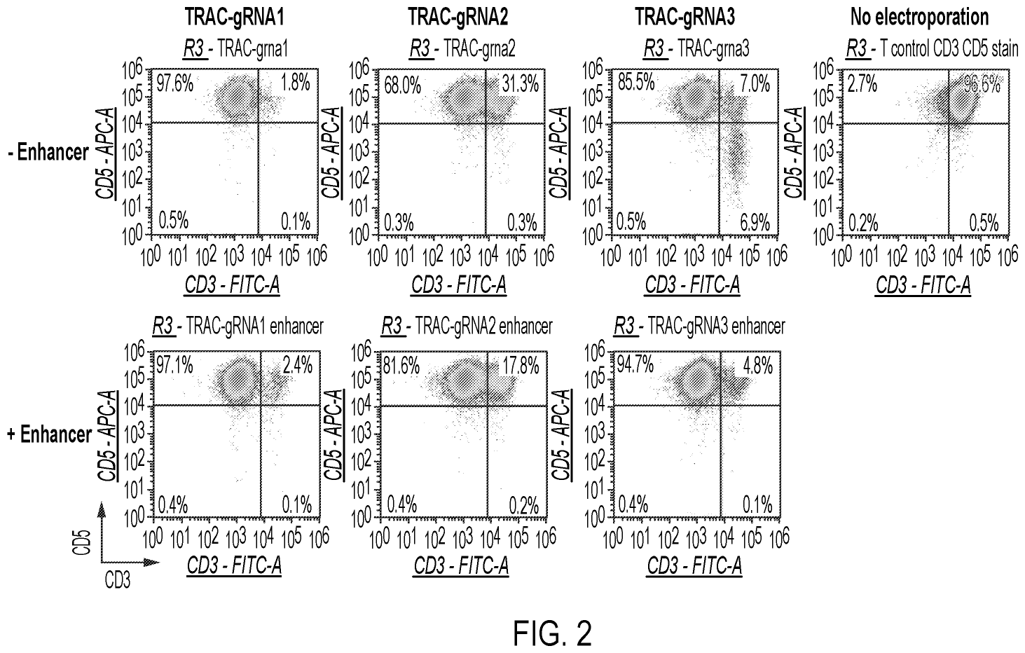

To delete T cell receptor (TCR), three gRNAs targeting on TRAC locus are screened (FIG. 2). Because TCR and CD3 form a complex on T cell surface, detection of CD3 expression indicates expression of TCR. TRAC-gRNA1 give the best knockout efficiency (>97%) based on reduction in CD3 expression. TRAC-gRNA3 gives rise to ˜95% KO efficiency in the presence of electroporation enhancer. TRAC-gRNA1 is chosen for future experiments.

CD5 Knockout gRNA Screen

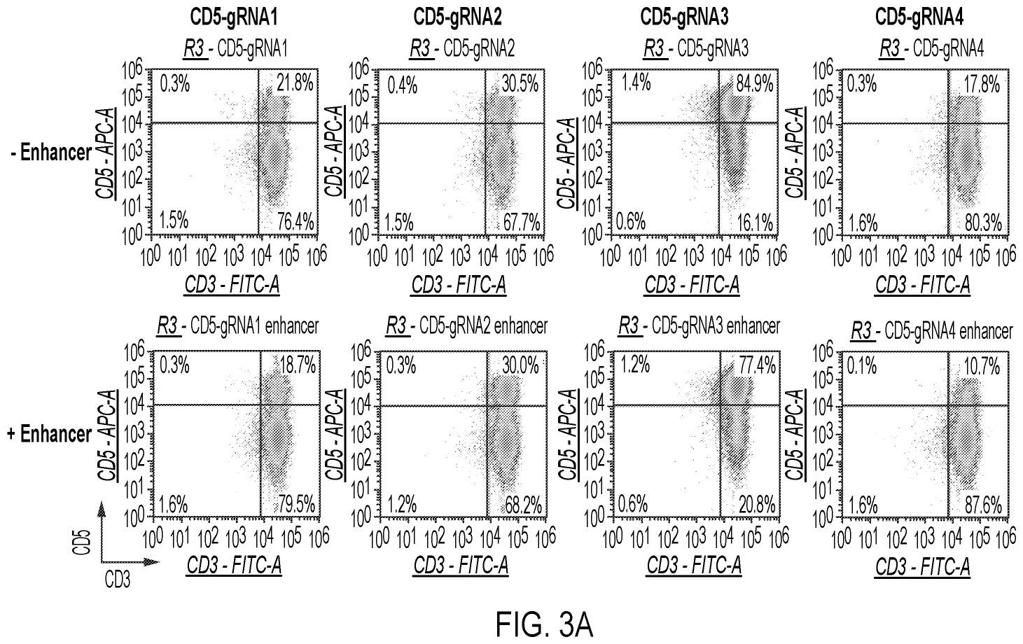

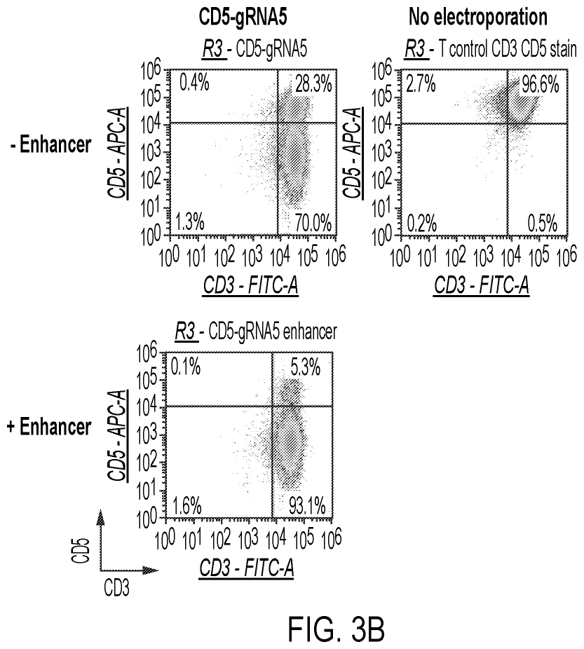

To delete CD5, 5 gRNAs are screened (FIGS. 3A-B). CD5-gRNA1, 4 and 5 could give more than 80% knockout efficiency, in which, CD5-gRNA5 could reach >90% KO efficiency based on reduction in CD5 expression in the presence of electroporation enhancer. CD5-gRNA5 is chosen for future experiment.

### CD5 CAR (CAR5) Screen: CD5 CAR Expression and Killing on Autologous T Cells

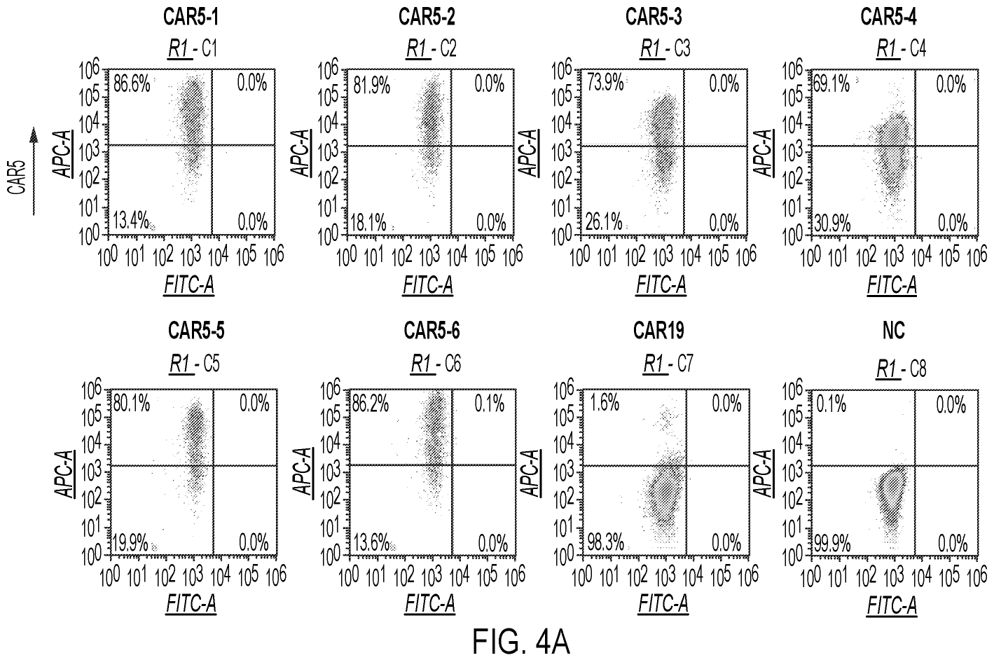

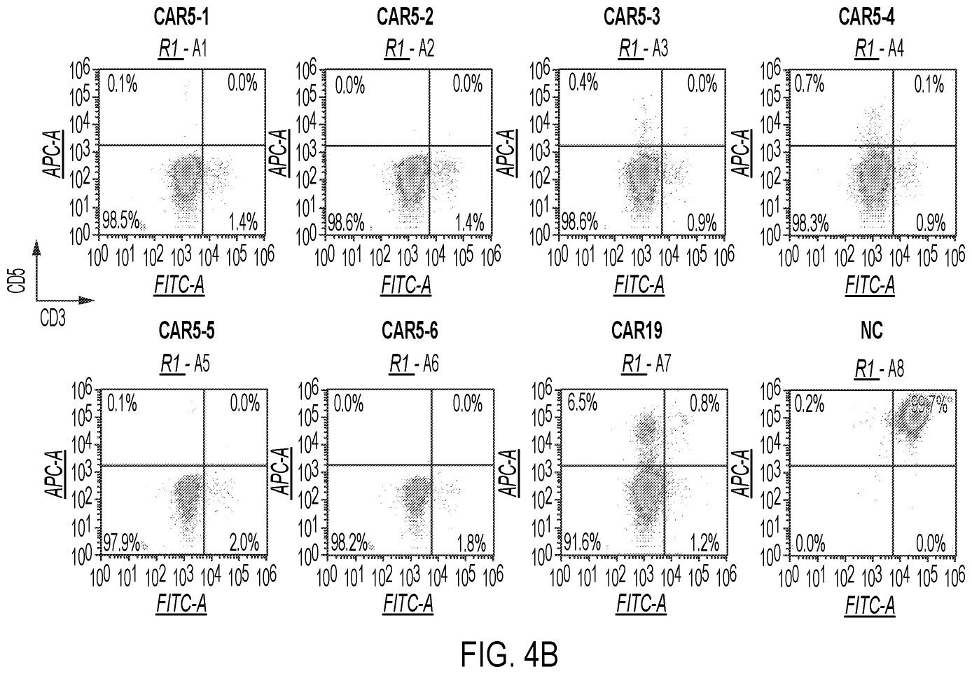

To screen for the best CAR5, six CAR5 constructs are screened (FIGS. 4A-B). T cells are activated for 2 days, and then electroporated with TRAC-RNP and CD5-RNP after removal of Dynabeads. The nucleofected cells are infected with CAR viral particles. The CAR5 expression reached more than 80% in CAR5-1, CAR5-2, CAR5-5, and CAR5-6, while in CAR5-3 and CAR5-4, CAR5 expression reached 70%.

Almost all the Pan-T cells express both CD3 and CD5. The knockout efficiencies of TRAC and CD5 are seen in CAR19 shown in FIG. 4B, where CD3-negative cell percentage is ˜98% and CD5-negative cell percentage is ˜92%. There are ˜7% of CD5-positive cells left after CD5 gene editing. In the cases of CAR5-1, -2, -5, and -6, CD5-positive cells disappeared, suggesting that CAR5 in those cases had strong fratricide activity against CD5-positive T cells. In CAR5-3 and -4, CD5-positive cells significantly reduced compared to the CAR19 case. The data indicates that CAR5 in these constructs had killing activity against autologous T cells.

### CD5 CAR (CAR5) Screen: Killing Activity of CAR5 on Allogeneic T Cells and T-ALL Cells

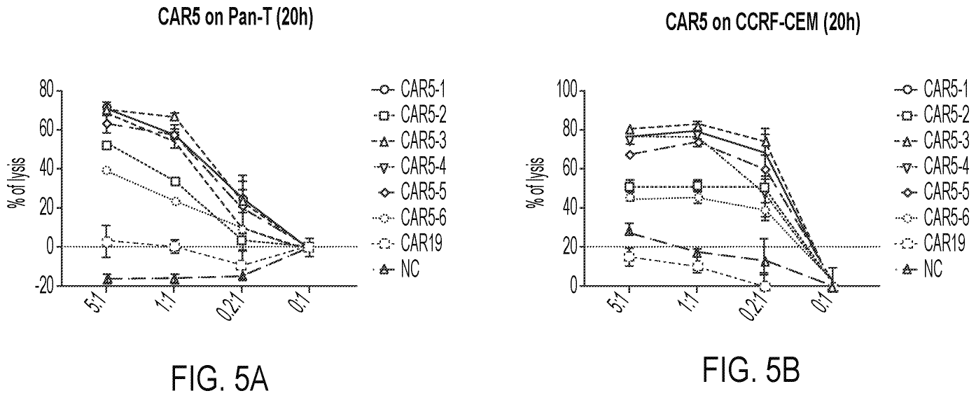

To study whether CAR5s also have killing activity on allogeneic T cells, the CAR5 T cells were cocultured with allogeneic Pan-T cells (from a different donor) labeled with CFSE at various E:T ratios. After 20 hours incubation, the cells are stained with DAPI and live allogeneic Pan-T cells are counted by flow cytometry. As shown in FIG. 5A, several CAR5 constructs had strong killing on allogeneic T cells. Next, CAR5 function is tested on T-ALL cell line (CCRF-CEM) and several constructs show clear killing activity on the T-ALL cells (FIG. 5B). Altogether, several of CAR5 constructs have strong killing activity against CD5 expressing cells including autologous and allogeneic T cells and T cell malignancy cell line. CAR5-1 and CAR5-5 are chosen for design of CD5/33 dual-CARs.

### CD33 CAR (CAR33) Screen

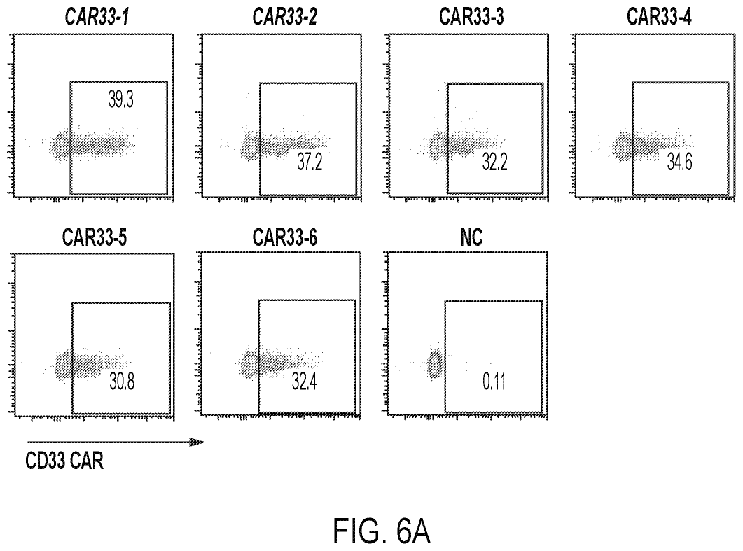

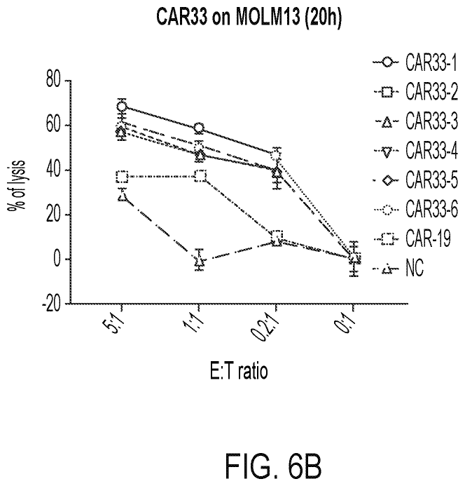

Six CAR33 constructs are designed, and the cells are infected with CAR33 lentivirus. The expression of CAR33 on T cells is shown in FIG. 6A. In CAR33-1 and -2, CAR33 expression shows separated populations, while CAR33 expression displays smear-like populations in the cases of CAR33-3 to CAR33-6. To test CAR33 function, AML cell line MOLM13 is used as a target cell line using CFSE-based flow assay (FIG. 6B). All six CAR33 constructs demonstrate higher killing activity against MOLM13 cells comparing with CAR19 and non-transduced T cells (NC). Due to their better expression, CAR33-1 and CAR33-2 are chosen for CD5/33 dual-CAR design and screening.

### CD5/33 Dual-CAR Design

There are multiple ways to express CD5/33 dual-CAR in T cells. In an embodiment, CAR5 and CAR33 are expressed on separate vectors and transduced to lymphocytes. In another embodiment, CAR5 and CAR33 are expressed in the same construct and two scFvs are linked in tandem (FIGS. 7E-F). In another embodiment, CAR5 and CAR33 are expressed in the same construct and two scFvs are linked in a loop (FIGS. 7A-D). In another embodiment, CAR5 and CAR33 are in the same construct and the two CARs are linked via a 2A peptide or IRES. Here the dual-CAR constructs are designed in the loop structure (FIGS. 7A-D) and in 2A linked as tandem structures (FIGS. 7E-F).

FIG. 7A illustrates the construct of S036 and S040. FIG. 7B illustrates the construct of S037 and S041. FIG. 7C illustrates the construct of S038 and S042. FIG. 7D illustrates the construct of S039 and S043. FIG. 7E illustrates the construct of S048. FIG. 7F illustrates the construct of S049.

### CD5/33 Dual-CAR Expression

To verify the CAR expression in the Pan-T cells after being transduced with the lentiviral-vector constructs encoding either single CAR5 (S005, S009), single CAR33 (S011, S012), or dual CAR5-CAR33 (S036, S037, S038, S039, S040, S041, S042, S043, S048, S049) with the corresponding scFv, levels of CAR expression on T cells are measured after 11 days post-transduction by staining with CD5 (CAR5) and CD33 (CAR33) antigen. CD19 CAR (S002) was used as negative control. Almost all the CAR-T samples express more than 70% of the CAR expression (CAR33) in either single or dual CAR constructs (FIG. 8).

CD5/33 dual-CAR function on cell line ectopically expressing CD5 or CD33

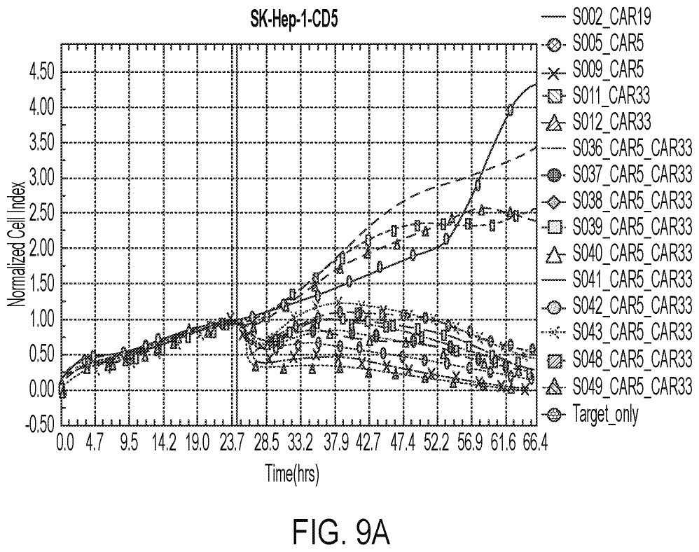

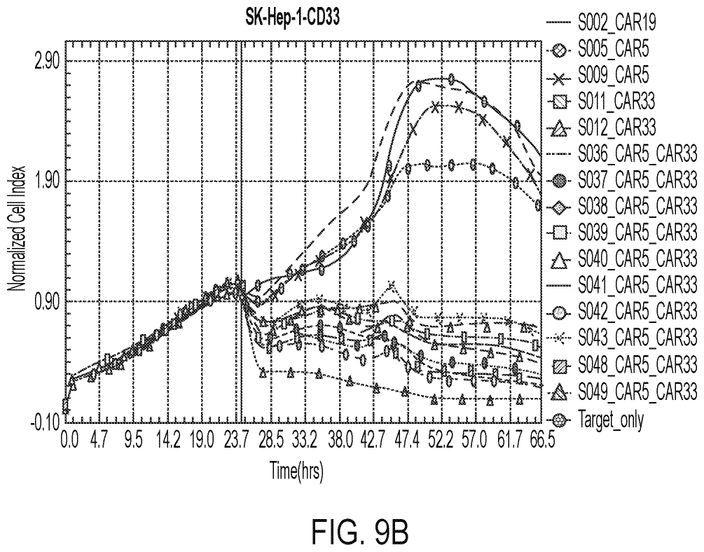

To examine the cytotoxic activity of the designed CAR constructs in vitro, SK-Hep-1 cells ectopically expressing CD5 or CD33 are used as target cell lines, and the cell number is measured by a real-time cellular impedance monitoring technology (RTCA). The impedance-related Cell Index (CI) parameter is measured, representing cell numbers for up to 42 hours after CAR-T cells are co-incubated with the cancer cells. The dual-CAR groups and single CAR5 groups show a significant killing effect, compared to the target only sample (SK-Hep1-CD5 without coculture with CAR-T cells), a control CAR (CAR19) and single CAR33 groups, when co-incubating with SK-Hep-1-CD5 cells (FIG. 9A). On the other hand, the dual-CAR constructs and single CAR33 groups show a significant killing effect when co-incubating with SK-Hep-1-CD33 cells, but not in the CAR19 or single CAR5 groups (FIG. 9B).

### CD5/33 Dual-CAR In Vitro Cytotoxicity Against CD33 Positive AML and T-ALL Cell Lines

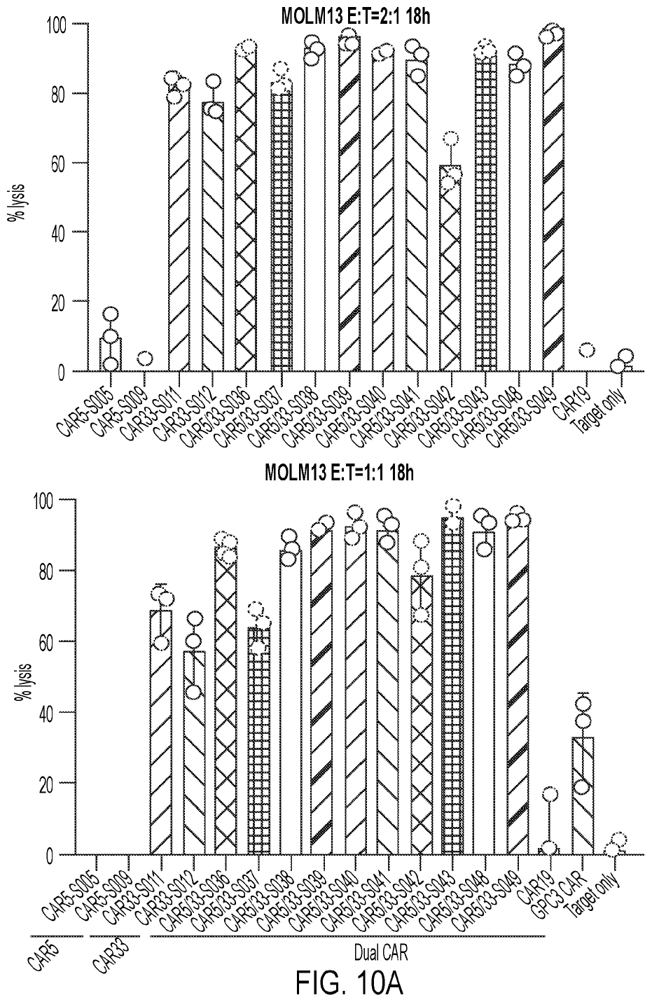

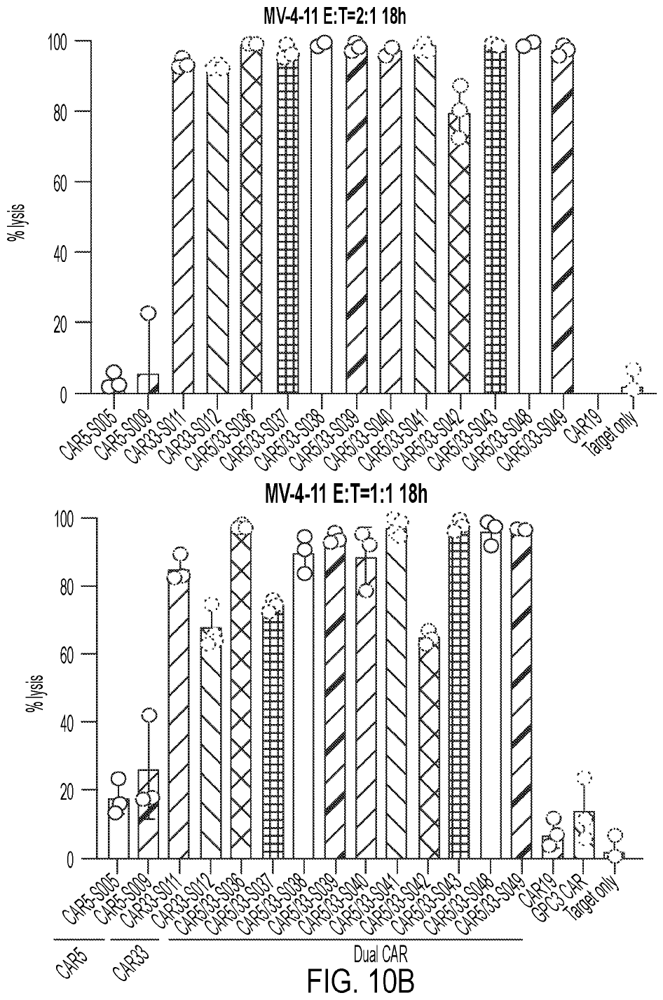

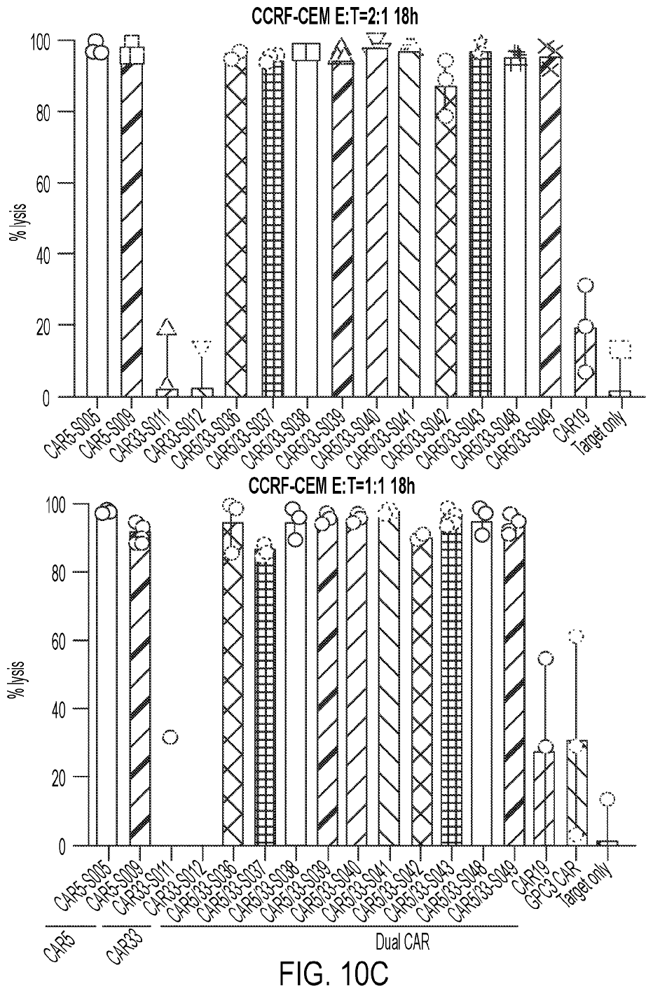

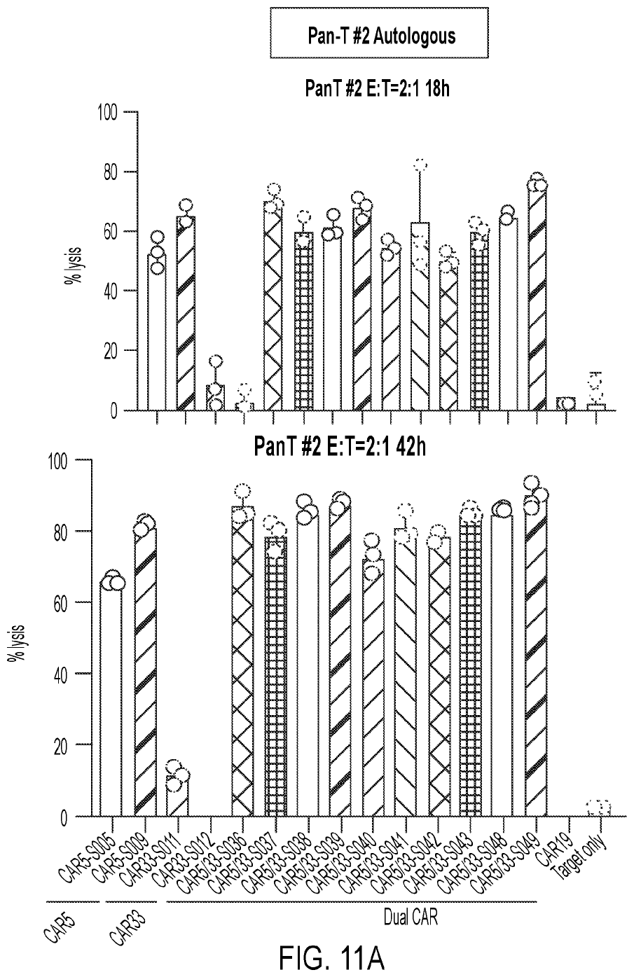

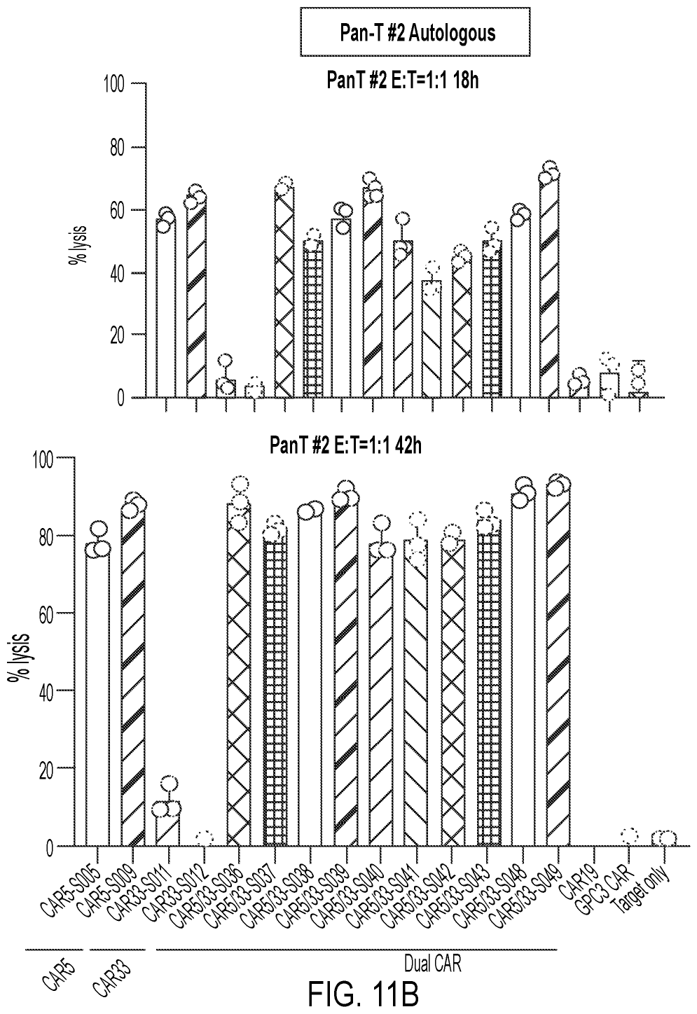

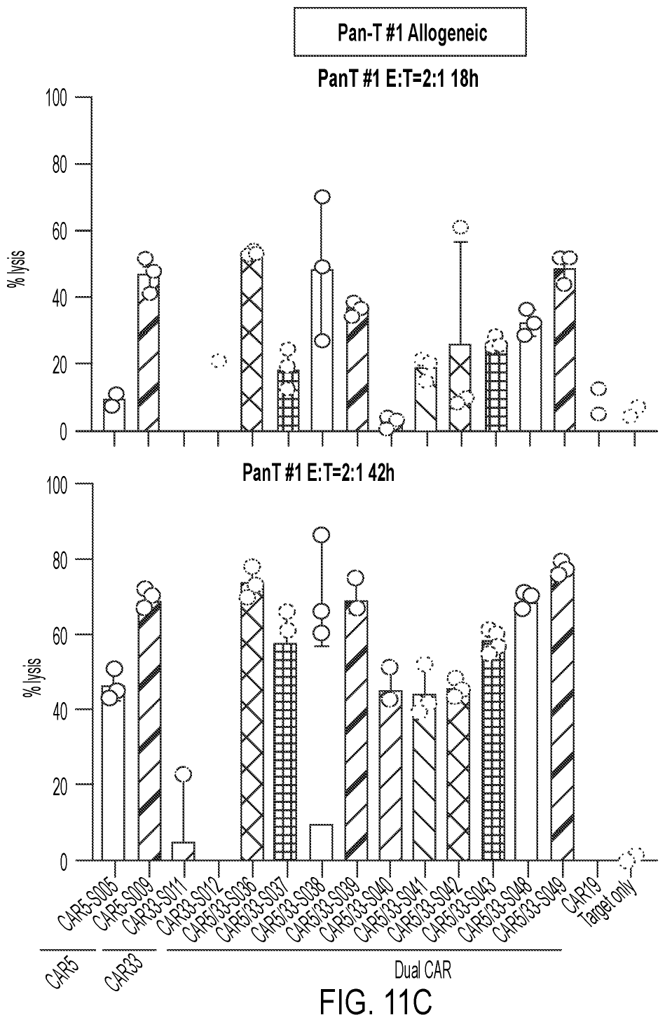

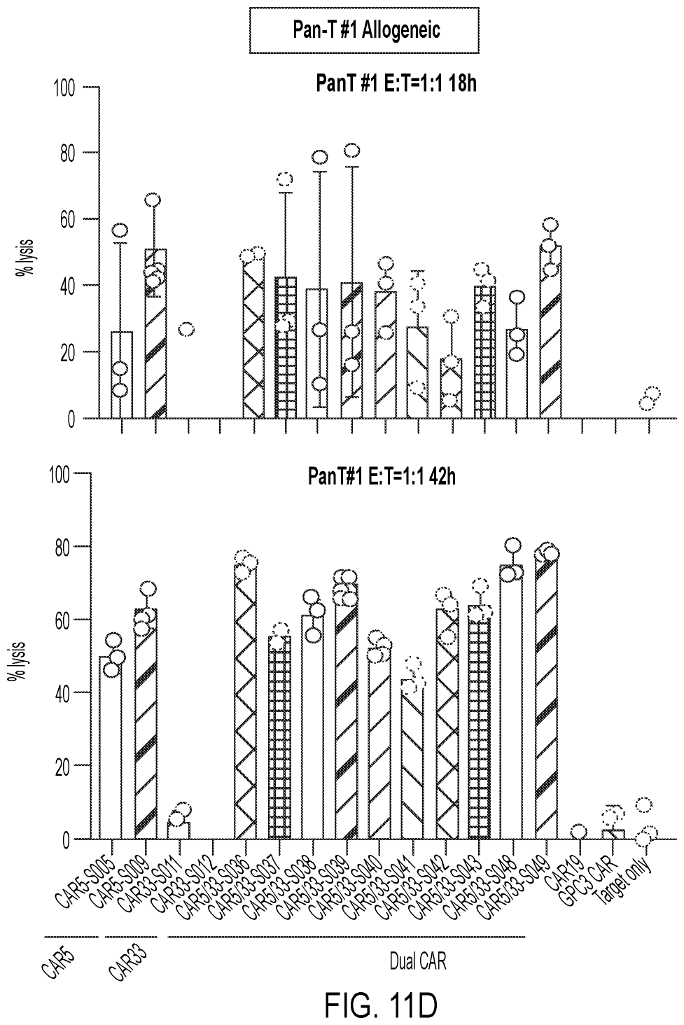

To assess the ability of CD5/CD33 dual-CART cells to eliminate tumor cells in vitro, a luciferase-based killing assay is performed on two acute myeloid leukemia (AML) cell lines—MOLM13 (CD33⁺) and MV-4-11 (CD33⁺), and a T lymphocytic leukemia (ALL) cell line CCRF-CEM (CD5⁺) (FIG. 10A-C). Incubation of CAR33 and CD5/CD33 dual-CAR T cells with MOLM13 (FIG. 10A) and MV-4-11 cell lines (FIG. 10B) results in 60˜100% of elimination after 18 hours in both E:T ratios. Some dual-CAR constructs show a higher killing activity compared to its single CAR controls at E:T ratio of 1:1. Incubation of CAR5 and CD5/CD33 dual-CAR T cells with CCRF-CEM cells eliminates almost 100% of the cancer cells after 18 hours (FIG. 10C). The data indicates that the dual-CAR constructs have a strong killing function on AML and T-ALL, and may completely eliminate these tumor cells.

### CD5/33 Dual-CAR can Eliminate Both Autologous and Allogeneic T Cells

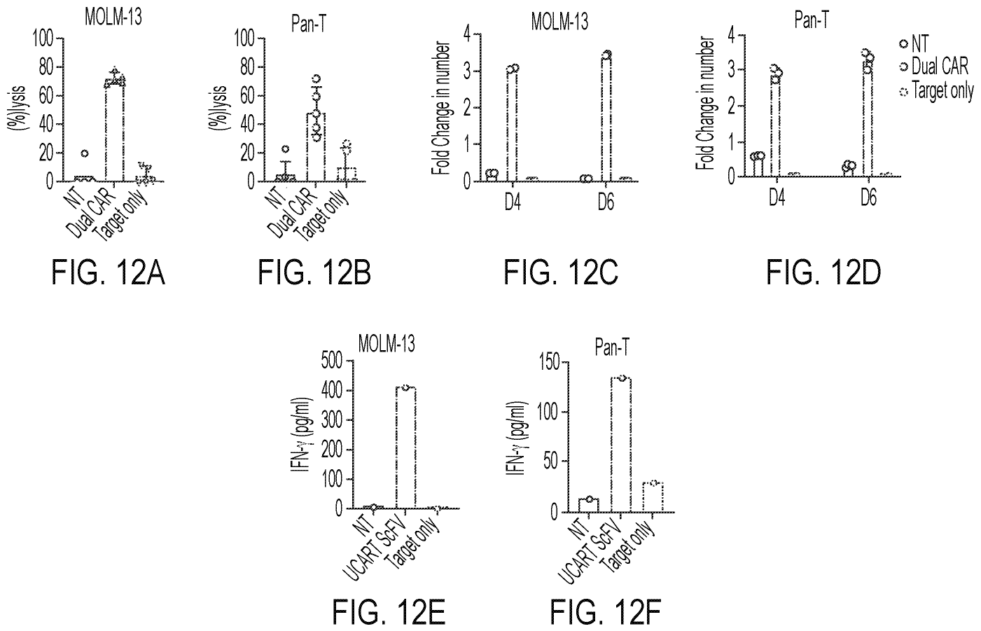

For allogeneic cell therapy, patient T cells are cleared using CAR5 in the dual-CAR constructs. First, dual-CAR constructs are tested for whether they could eliminate autologous T cells expressing CD5. Autologous Pan-T cells are labeled with CFSE and co-incubated with CAR-T cells for up to 42 hours with two E:T ratios (2:1 and 1:1). Some of dual-CAR constructs show 70˜90% killing activity at 42 hours, similar to the activity of those single CAR5 constructs (FIG. 12A). The dual-CAR constructs also show strong killing activity on allogeneic T cells, some of which eliminate approximately 70% of allogeneic Pan-T cells within two days (FIG. 12B).

CD5 (CAR5) can be Protected from Rejection

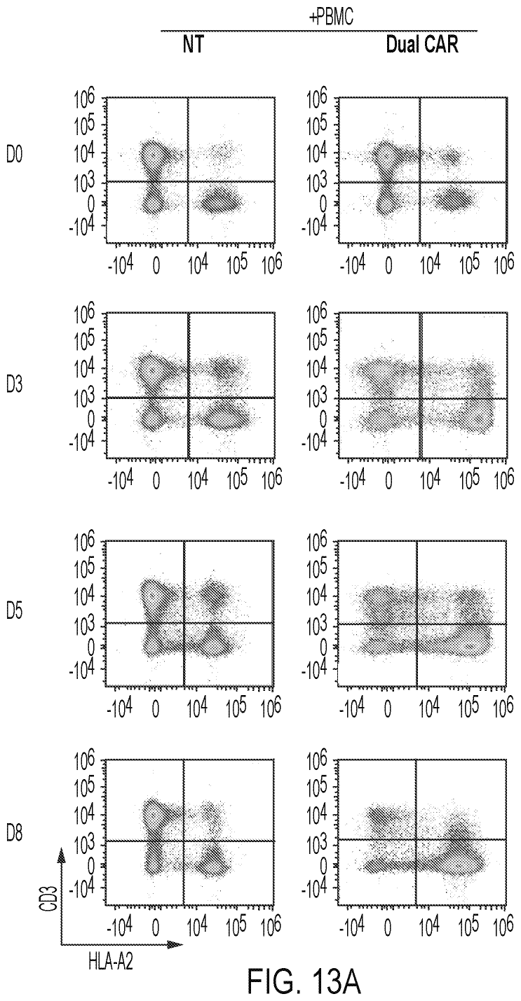

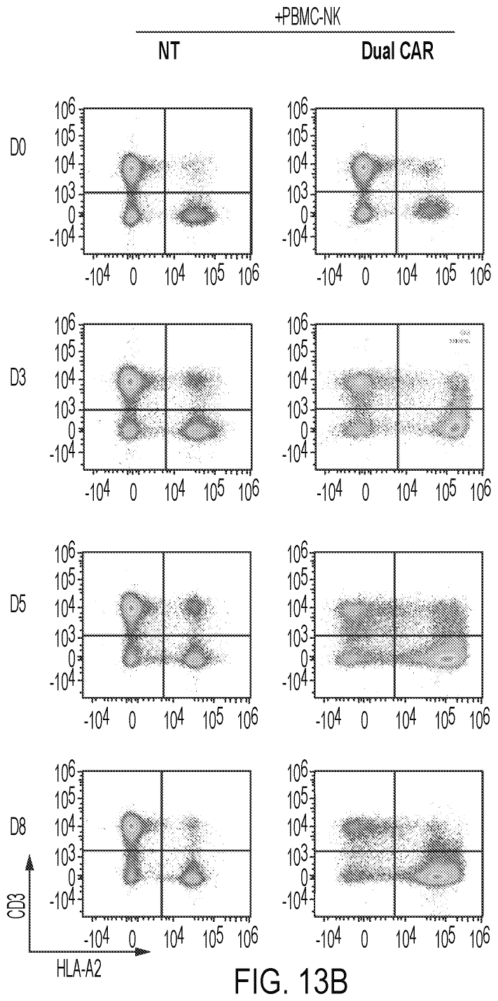

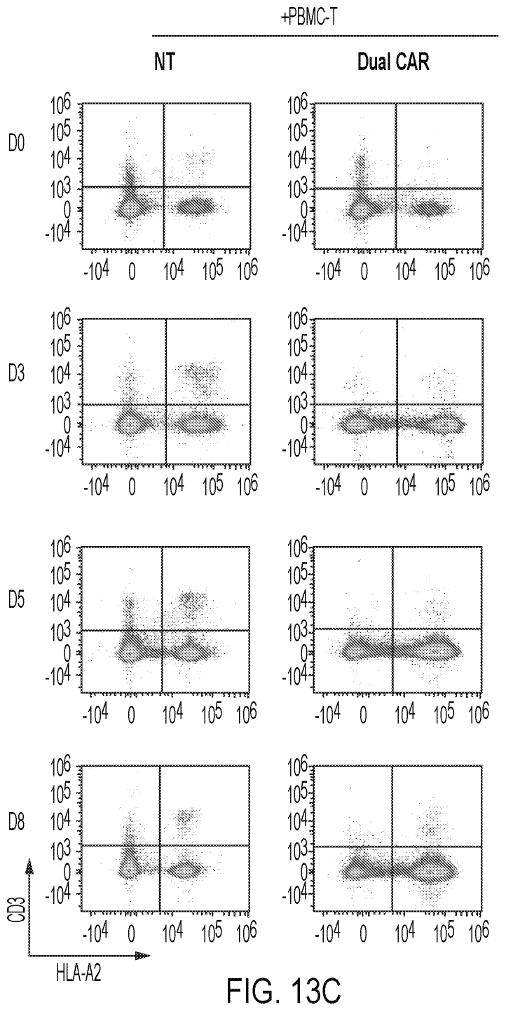

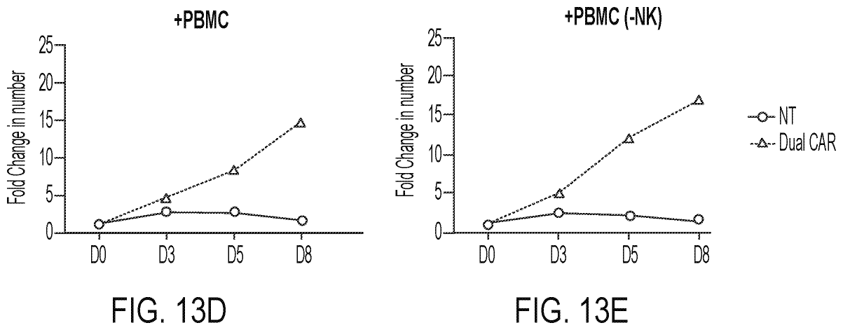

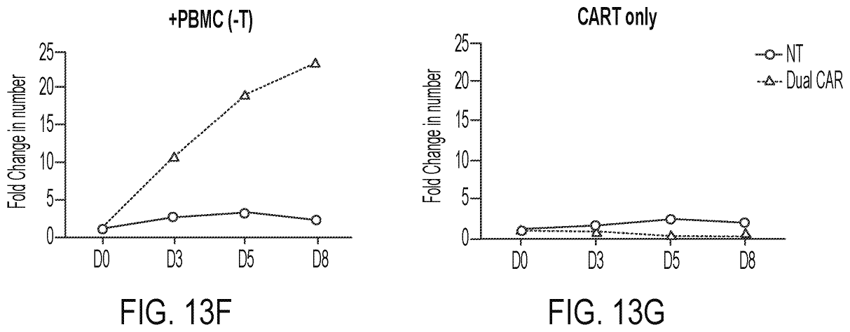

To determine whether CAR5 T cells can be protected from T cell based allogeneic HVGD rejection, CAR5-1 was transduced by using HLA-A2+ donor and coculture with HLA-A2⁻ PBMC alone, PBMC depleted with NK cells by CD56 beads, or PBMC depleted with T cells by CD3 beads for either 0, 3, 5, or 7 days (FIGS. 13A-C). Cells were further counted by flow cytometry and used counting beads to normalize cell numbers. FIGS. 13D-G represent the fold changes of the cell number for the experiment of FIGS. 13A-C, indicating in each day relative to the number from Day 0. Based on the result, CAR5 can complete deplete T cells based on the CD3 positive cell numbers, and at the meantime there are more cells maintain at the D3 (FIGS. 13D-G).

### Cytokine Release and Cell Proliferation Assay

CD5/CD33 dual CAR T cells were coculture with AML cells lines (molm13), T leukemia cells (CCRF), or allogenic T cells. Supernatants were collected 24 hours after coculture (FIGS. 12E-F). Cytokine release (INF-gamma) were detected with ELISA kit (Biolegend CAT #430804). T cell number were accounted 5 days after coculture.

### CD5/33 Dual-CAR can Retard Tumor Progression In Vivo

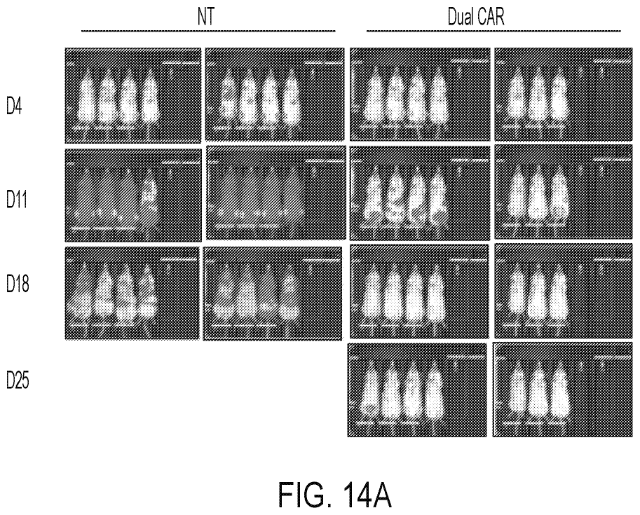

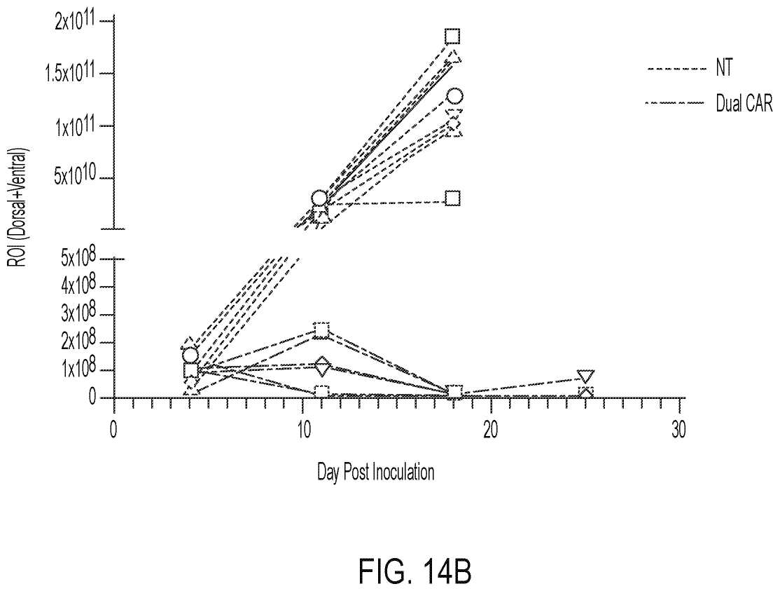

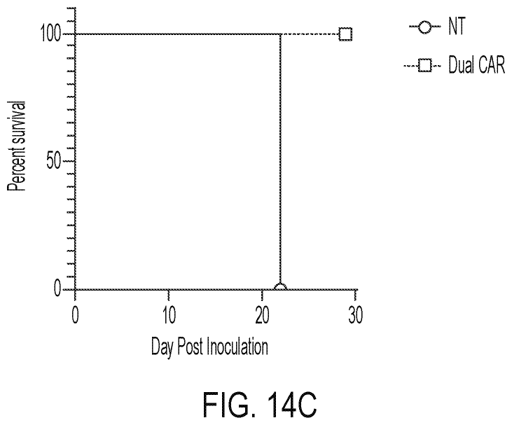

In order to determine the functional activities of the dual-CAR constructs in vivo, MOLM13-luc (luciferase) cell lines and allogeneic T cells (HLA-A2 negative) were injected 5 days prior to CD5/CD33 dual CAR+ T cells (HLA-A2 positive) injection (i.v.) in the immunodeficient NOD/SCID/IL-2Rγc^{null} (NSG) mice. Tumor growth was monitored weekly by bioluminescence imaging (FIGS. 14A-B). Animals' survival curve was analyzed using Kaplan-Meier method (FIG. 14C).

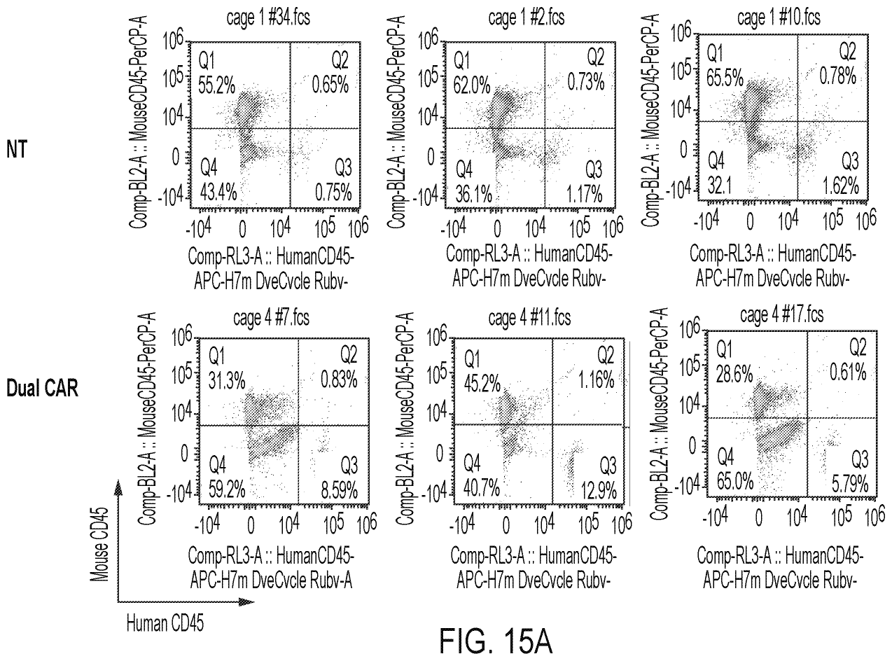

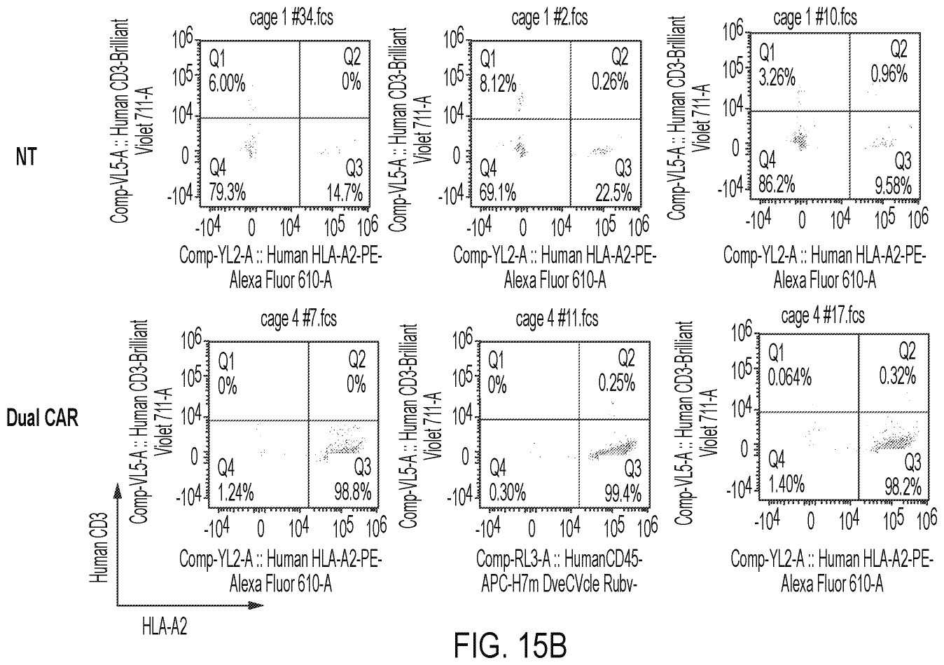

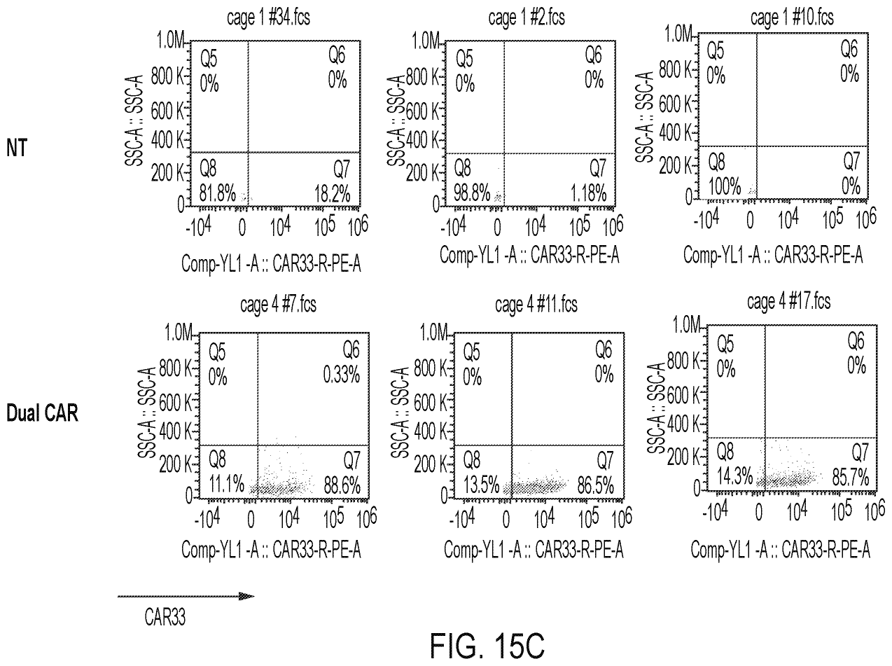

Blood samples from mice were collected for flow cytometric analyzing CAR-T cell persistence and expansion. Blood samples were stained with anti-mouse CD45, anti-human CD45, anti-human HLA-A2, anti-human CD3 and CD33 antigen for 30 min at RT and then red blood cells were eliminated with blood lysing buffer. Samples were analyzed on flow cytometry and data were analyzed with FlowJo software v10.6.2 (FIGS. 15A-C).

While example embodiments have been particularly shown and described, it will be understood by those skilled in the art that various changes in form and details may be made therein without departing from the scope of the embodiments encompassed by the appended claims.

1. A transgenic lymphocyte that expresses a chimeric antigen receptor (CAR), the CAR comprising:
a) a signal peptide domain;
b) an extracellular domain comprising variable light (V_{L}) and variable heavy (V_{H}) domains that bind cluster differentiation 5 (CD5), V_{L} and V_{H} domains that bind cluster differentiation 33 (CD33), and a linker domain between adjacent V_{L} and V_{H} domains;
c) a hinge domain;
d) a transmembrane domain; and
e) a costimulatory domain.

2. The transgenic lymphocyte of claim 1, wherein the signal peptide is:
(1) N-terminal to the V_{L} domain that binds CD5, which is N-terminal to the V_{H} domain that binds CD33, which is N-terminal to the V_{L} domain that binds CD33, which is N-terminal to the V_{H} domain that binds CD5;
(2) N-terminal to the V_{H} domain that binds CD5, which is N-terminal to the V_{L} domain that binds CD33, which is N-terminal to the V_{H} domain that binds CD33, which is N-terminal to the V_{L} domain that binds CD5;
(3) N-terminal to the V_{L} domain that binds CD33, which is N-terminal to the V_{H} domain that binds CD5, which is N-terminal to the V_{L} domain that binds CD5, which is N-terminal to the V_{H} domain that binds CD33; or
(4) N-terminal to the V_{H} domain that binds CD33, which is N-terminal to the V_{L} domain that binds CD5, which is N-terminal to the V_{H} domain that binds CD5, which is N-terminal to the V_{L} domain that binds CD33.

3-5. (canceled)

6. The transgenic lymphocyte of claim 1, wherein the signal peptide is a CD8α signal peptide, a GM-CSF signal peptide, a CD4 signal peptide, a CD137 (4-1BB) signal peptide, or a combination thereof;
wherein one or more of the linker domains is (G4S)n, wherein n is 1 or 3, a 218 linker, or a combination thereof;
wherein the hinge domain is a CD8α hinge domain, a CD28 hinge domain, a CD137 hinge domain, an IgG1 hinge domain, an IgG2 hinge domain, an IgG3 hinge domain, an IgG4 hinge domain, or a combination thereof,
wherein the transmembrane domain is a CD8α transmembrane domain, a CD28 transmembrane domain, a CD3e transmembrane domain, a CD45 transmembrane domain, a CD4 transmembrane domain, a CD5 transmembrane domain, a CD9 transmembrane domain, a CD16 transmembrane domain, a CD22 transmembrane domain, a CD33 transmembrane domain, a CD37 transmembrane domain, a CD64 transmembrane domain, a CD80 transmembrane domain, a CD86 transmembrane domain, a CD134 transmembrane domain, a CD137 transmembrane domain, or a transmembrane domain CD154, or
wherein the costimulatory domain is a 4-1BB costimulatory domain, a CD28 costimulatory domain, a OX40 costimulatory domain, a CD2 costimulatory domain, a CD7 costimulatory domain, a CD27 costimulatory domain, a CD28 costimulatory domain, a CD30 costimulatory domain, a CD40 costimulatory domain, a CD70 costimulatory domain, a CD134 costimulatory domain, a PD1 costimulatory domain, an ICOS costimulatory domain, an NKG2D costimulatory domain, a GITR costimulatory domain, or a TLR2 costimulatory domain.

7-10. (canceled)

11. The transgenic lymphocyte of claim 1, wherein the V_{H} domain that binds CD5 comprises an amino acid sequence that is at least 95% identical to any of SEQ ID NOs: 26-31.

12-16. (canceled)

17. The transgenic lymphocyte of claim 1, wherein the CAR comprises an amino acid sequence that is at least 95% identical to any of SEQ ID NOs: 14-23,
wherein the CAR comprises an amino acid sequence selected from the group consisting of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 and SEQ ID NO: 23.

18-27. (canceled)

28. The transgenic lymphocyte of claim 1, wherein the transgenic lymphocyte is a T cell,
wherein the T cell expresses T-cell receptor alpha chain or T-cell receptor beta chain at a level that does not elicit a graft versus host disease (GVHD) response when the transgenic lymphocyte is administered to a patient.

29. (canceled)

30. The transgenic lymphocyte of claim 1, wherein the transgenic lymphocyte is a natural killer (NK) cell.

31. The transgenic lymphocyte of claim 1, wherein the transgenic lymphocyte expresses an exogenous nucleic acid encoding a cytokine or a cytokine receptor gene, or an exogenous nucleic acid encoding a suicide gene;
the encoded cytokine or cytokine receptor gene is interleukin 2, interleukin 7, interleukin 12, interleukin 15, or interleukin 21.

32-57. (canceled)

58. A method of making a transgenic lymphocyte according to claim 1, the method comprising introducing a nucleic acid into a lymphocyte, the nucleic acid encoding a chimeric antigen receptor (CAR) comprising:
a) a signal peptide domain;
b) an extracellular domain comprising variable light (V_{L}) and variable heavy (V_{H}) domains that bind cluster differentiation 5 (CD5), V_{L} and V_{H} domains that bind cluster differentiation 33 (CD33), and a linker domain between adjacent V_{L} and V_{H} domains;
c) a hinge domain;
d) a transmembrane domain; and
e) a costimulatory domain.

59. The method of claim 58, wherein introducing the nucleic acid into the lymphocyte comprises electroporation, transformation, or transduction.

60. The method of claim 58, wherein the nucleic acid is introduced by a viral vector, a non-viral vector, or naked DNA;
wherein the viral vector is a lentivirus vector or an adeno-associated virus vector.

61. (canceled)

62. The method of claim 58, wherein the nucleic acid is integrated into the lymphocyte genome;
wherein the nucleic acid is randomly integrated in the lymphocyte genome.

63. (canceled)

64. The method of claim 58, wherein the method is performed under conditions that allow expression of the CAR in the transgenic lymphocyte.

65. The method of claim 58, wherein the cell expresses the CAR upon introduction of the nucleic acid.

66-74. (canceled)

75. The method of claim 58, wherein the method comprising introducing first and second nucleic acids into a lymphocyte, wherein the first nucleic acid encodes the CAR that binds CD5, and wherein the CAR that binds CD5 comprises:
a) a signal peptide domain;
b) an extracellular domain comprising a variable light (V_{L}) and variable heavy (V_{H}) domains that bind CD5;
c) a hinge domain;
d) a transmembrane domain; and
e) a costimulatory domain; and
wherein the second nucleic acid encodes the CAR that binds CD33, and wherein the CAR that binds CD33 comprises:
f) a signal peptide domain;
g) an extracellular domain comprising variable light (V_{L}) and variable heavy (V_{H}) domains that bind to CD33;
h) a hinge domain;
i) a transmembrane domain; and
j) a costimulatory domain;
wherein the CAR that binds CD5 and the CAR that binds CD33 are joined by a self-cleaving peptide, wherein the self-cleaving peptide is a 2A peptide.

76. A nucleic acid encoding a chimeric antigen receptor (CAR) as defined in claim 1, wherein the chimeric antigen receptor comprising:
a) a signal peptide domain;
b) an extracellular domain comprising variable light (V_{L}) and variable heavy (V_{H}) domains that bind cluster differentiation 5 (CD5), V_{L} and V_{H} domains that bind cluster differentiation 33 (CD33), and a linker domain between adjacent V_{L} and V_{H} domains;
c) a hinge domain;
d) a transmembrane domain; and
e) a costimulatory domain.

77. (canceled)

78. A method of treating acute myeloid leukemia (AML), the method comprising administering to a patient in need thereof a therapeutically effective amount of a transgenic lymphocyte according to claim 1, wherein the AML comprises leukemic cells that express CD33 as a cell-surface protein.

79. The method of claim 78, wherein the transgenic lymphocyte is allogeneic or autologous.

80-81. (canceled)

82. A method of treating a T-cell malignancy, the method comprising administering to a patient in need thereof a therapeutically effective amount of a transgenic lymphocyte according to claim 1;
wherein the T-cell malignancy comprises T-cells that express CD5 as a cell-surface protein.

83. The method of claim 82, wherein the transgenic lymphocyte is allogeneic or autologous.

84-85. (canceled)


SEQUENCE LISTING (44 sequences)

SEQ ID NO: 1 - protein, 491 aa, Artificial Sequence
  S002, CAR19
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 486 aa, Artificial Sequence
  S005, CAR5-1
MALPVTALLLPLALLLHAARPDIKMTQSPSSMYASLGERVTITCKASQDINSYLSWFHHKPGKSPKTLIYRANRLVDGVPSRFSGSGSGQDYSLTISSLDYEDMGIYYCQQYDESPWTFGGGTKLEMKGGGGSGGGGSGGGGSNIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 486 aa, Artificial Sequence
  S006, CAR5-2
MALPVTALLLPLALLLHAARPNIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSGGGGSGGGGSGGGGSDIKMTQSPSSMYASLGERVTITCKASQDINSYLSWFHHKPGKSPKTLIYRANRLVDGVPSRFSGSGSGQDYSLTISSLDYEDMGIYYCQQYDESPWTFGGGTKLEMKASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 486 aa, Artificial Sequence
  S007, CAR5-3
MALPVTALLLPLALLLHAARPDIQVTQSPSSLSASLGERISLTCRTSQDISNYLNWFQQKPDGTFKRLIYATSSLDSGVPKRFSGSGSGSDYSLTISSLESEDFADYYCLQYASYPFTFGSGTKLEIKGGGGSGGGGSGGGGSEVQLQESGPGLVKPSQTLSLTCSVTGYSITSGYYWHWIRQFPGNKLQWMGYISYSGFTNYKTSLINRISITHDTSENQFFLNLNSVTTEDTATYYCAGDRTGSWFAYWGQGTLVTVSAASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 486 aa, Artificial Sequence
  S008, CAR5-4
MALPVTALLLPLALLLHAARPEVQLQESGPGLVKPSQTLSLTCSVTGYSITSGYYWHWIRQFPGNKLQWMGYISYSGFTNYKTSLINRISITHDTSENQFFLNLNSVTTEDTATYYCAGDRTGSWFAYWGQGTLVTVSAGGGGSGGGGSGGGGSDIQVTQSPSSLSASLGERISLTCRTSQDISNYLNWFQQKPDGTFKRLIYATSSLDSGVPKRFSGSGSGSDYSLTISSLESEDFADYYCLQYASYPFTFGSGTKLEIKASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 486 aa, Artificial Sequence
  S009, CAR5-5
MALPVTALLLPLALLLHAARPDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKGGGGSGGGGSGGGGSEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 486 aa, Artificial Sequence
  S010, CAR5-6
MALPVTALLLPLALLLHAARPEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 488 aa, Artificial Sequence
  S011, CAR33-1
MALPVTALLLPLALLLHAARPDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGGGSGGGGSGGGGSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 488 aa, Artificial Sequence
  S012, CAR33-2
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 492 aa, Artificial Sequence
  S013, CAR33-3
MALPVTALLLPLALLLHAARPEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGGGGSGGGGSGGGGSQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 492 aa, Artificial Sequence
  S014, CAR33-4
MALPVTALLLPLALLLHAARPQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSGGGGSGGGGSGGGGSEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 492 aa, Artificial Sequence
  S015, CAR33-5
MALPVTALLLPLALLLHAARPNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRGGGGSGGGGSGGGGSQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 492 aa, Artificial Sequence
  S016, CAR33-6
MALPVTALLLPLALLLHAARPQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSGGGGSGGGGSGGGGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 726 aa, Artificial Sequence
  S036, DUAL-CAR
MALPVTALLLPLALLLHAARPDIKMTQSPSSMYASLGERVTITCKASQDINSYLSWFHHKPGKSPKTLIYRANRLVDGVPSRFSGSGSGQDYSLTISSLDYEDMGIYYCQQYDESPWTFGGGTKLEMKGGGGSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGSTSGSGKPGSGEGSTKGDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGGGSNIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 726 aa, Artificial Sequence
  S037, DUAL-CAR
MALPVTALLLPLALLLHAARPNIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGSTSGSGKPGSGEGSTKGQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSDIKMTQSPSSMYASLGERVTITCKASQDINSYLSWFHHKPGKSPKTLIYRANRLVDGVPSRFSGSGSGQDYSLTISSLDYEDMGIYYCQQYDESPWTFGGGTKLEMKASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 726 aa, Artificial Sequence
  S038, DUAL-CAR
MALPVTALLLPLALLLHAARPDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGGGSNIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSGSTSGSGKPGSGEGSTKGDIKMTQSPSSMYASLGERVTITCKASQDINSYLSWFHHKPGKSPKTLIYRANRLVDGVPSRFSGSGSGQDYSLTISSLDYEDMGIYYCQQYDESPWTFGGGTKLEMKGGGGSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 726 aa, Artificial Sequence
  S039, DUAL-CAR
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSDIKMTQSPSSMYASLGERVTITCKASQDINSYLSWFHHKPGKSPKTLIYRANRLVDGVPSRFSGSGSGQDYSLTISSLDYEDMGIYYCQQYDESPWTFGGGTKLEMKGSTSGSGKPGSGEGSTKGNIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 726 aa, Artificial Sequence
  S040, DUAL-CAR
MALPVTALLLPLALLLHAARPDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKGGGGSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGSTSGSGKPGSGEGSTKGDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGGGSEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 726 aa, Artificial Sequence
  S041, DUAL-CAR
MALPVTALLLPLALLLHAARPEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGSTSGSGKPGSGEGSTKGQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 726 aa, Artificial Sequence
  S042, DUAL-CAR
MALPVTALLLPLALLLHAARPDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGGGSEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGSTSGSGKPGSGEGSTKGDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKGGGGSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 726 aa, Artificial Sequence
  S043, DUAL-CAR
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKGSTSGSGKPGSGEGSTKGEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 998 aa, Artificial Sequence
  S048, DUAL-CAR
MALPVTALLLPLALLLHAARPDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGGGSGGGGSGGGGSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIKMTQSPSSMYASLGERVTITCKASQDINSYLSWFHHKPGKSPKTLIYRANRLVDGVPSRFSGSGSGQDYSLTISSLDYEDMGIYYCQQYDESPWTFGGGTKLEMKGGGGSGGGGSGGGGSNIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRFE

SEQ ID NO: 23 - protein, 998 aa, Artificial Sequence
  S049, DUAL-CAR
MALPVTALLLPLALLLHAARPDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGGGSGGGGSGGGGSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKGGGGSGGGGSGGGGSEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRFE

SEQ ID NO: 24 - protein, 758 aa, Artificial Sequence
  S026, CD5-GFP
MPMGSLQPLATLYLLGMLVASCLGRLSWYDPDFQARLTRSNSKCQGQLEVYLKDGWHMVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNPAGLAAGTVASIILALVLLVVLLVVCGPLAYKKLVKKFRQKKQRQWIGPTGMNQNMSFHRNHTATVRSHAENPTASHVDNEYSQPPRNSHLSAYPALEGALHRSSMQPDNSSDSDYDLHGAQRLFEGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 25 - protein, 627 aa, Artificial Sequence
  S027, CD33-GFP
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQFEGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 26 - protein, 118 aa, Artificial Sequence
  CD5 ScFV V.sub.H
NIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSS

SEQ ID NO: 27 - protein, 107 aa, Artificial Sequence
  CD5 ScFV V.sub.L
DIKMTQSPSSMYASLGERVTITCKASQDINSYLSWFHHKPGKSPKTLIYRANRLVDGVPSRFSGSGSGQDYSLTISSLDYEDMGIYYCQQYDESPWTFGGGTKLEMK

SEQ ID NO: 28 - protein, 118 aa, Artificial Sequence
  CD5 ScFV V.sub.H
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSS

SEQ ID NO: 29 - protein, 107 aa, Artificial Sequence
  CD5 ScFV V.sub.L
DIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 30 - protein, 116 aa, Artificial Sequence
  CD33 ScFV V.sub.H
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 31 - protein, 111 aa, Artificial Sequence
  CD33 ScFV V.sub.L
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 32 - DNA, 20 bp, Artificial Sequence
  TRAC-gRNA1
ttcggaacccaatcactgac

SEQ ID NO: 33 - DNA, 20 bp, Artificial Sequence
  TRAC-gRNA2
aagttcctgtgatgtcaagc

SEQ ID NO: 34 - DNA, 20 bp, Artificial Sequence
  TRAC-gRNA3
tcagggttctggatatctgt

SEQ ID NO: 35 - DNA, 20 bp, Artificial Sequence
  CD5-gRNA1
agcggttgcagagaccccat

SEQ ID NO: 36 - DNA, 20 bp, Artificial Sequence
  CD5-gRNA2
tggccaccttgtacctgctg

SEQ ID NO: 37 - DNA, 20 bp, Artificial Sequence
  CD5-gRNA3
ctggcacttcgagttggaac

SEQ ID NO: 38 - DNA, 20 bp, Artificial Sequence
  CD5-gRNA4
gcttcaagaaggagccacac

SEQ ID NO: 39 - DNA, 20 bp, Artificial Sequence
  CD5-gRNA5
gagccttgcctggaaatctg

SEQ ID NO: 40 - protein, 21 aa, Artificial Sequence
  CD8.alpha. signal peptide
MALPVTALLLPLALLLHAARP

SEQ ID NO: 41 - protein, 17 aa, Artificial Sequence
  Granulocyte-macrophage colony-stimulating factor (GM-CSF) signal peptide
MWLQSLLLLGTVACSIS

SEQ ID NO: 42 - protein, 25 aa, Artificial Sequence
  CD4 signal peptide
MNRGVPFRHLLLVLQLALLPAATQG

SEQ ID NO: 43 - protein, 23 aa, Artificial Sequence
  CD137 (4-1BB) signal peptide
MGNSCYNIVATLLLVLNFERTRS

SEQ ID NO: 44 - protein, 18 aa, Artificial Sequence
  218 linker
GSTSGSGKPGSGEGSTKG